(19) United States
(12) Patent Application Publication
(10) Pub. No.: US 2020/0316159 A1
(43) Pub. Date: Oct. 8, 2020

(54) SUSTAINED RELEASE COMPOSITIONS OF KAPPA-OPIOID RECEPTOR AGONIST

(71) Applicant: JT PHARMACEUTICALS, INC., Mount Pleasant, SC (US)

(72) Inventor: Gautam GHATNEKAR, Mount Pleasant, SC (US)

(21) Appl. No.: 16/305,209

(22) PCT Filed: Jun. 5, 2017
(86) PCT No.: PCT/US2017/035874

§ 371 Date: Nov. 28, 2018

Related U.S. Application Data
(60) Provisional application No. 62/345,583, filed on Jun. 3, 2016.

(51) Int. Cl. A61K 38/07; A61K 9/00; A61P 23/00

(52) U.S. Cl. CPC A61K 38/07; A61P 23/00; A61K 9/0024

(57) ABSTRACT

Disclosed herein are methods and compositions for sustained release of kappa-opioid agonists. The modified kappa-opioid agonists disclosed herein exhibit high peripheral to CNS selectivity, and benefits patients with visceral and neuropathic pain. In some embodiments, these kappa-opioid agonists of formula I are highly specific for kappa receptors with little or no agonist or antagonist activity to mu or delta receptors. In some embodiments, the kappa-opioid agonists of formula I do not cause CNS-dependent adverse effects. The kappa-opioid agonists of formula I may not cross blood-brain barrier to elicit side effects.

This application is a national stage application of PCT Application No. PCT/US2017/035874, filed on Jun. 5, 2017, which claims priority to U.S. Provisional Application No. 62/345,583 filed on Jun. 3, 2016 and each of these disclosures is incorporated herein by reference in its entirety.

### SUMMARY

Disclosed herein are compositions for sustained release of kappa-opioid agonists and methods of using the same. In one embodiment, a sustained release composition includes a biocompatible polymeric matrix and a kappa-opioid receptor agonist of formula I:

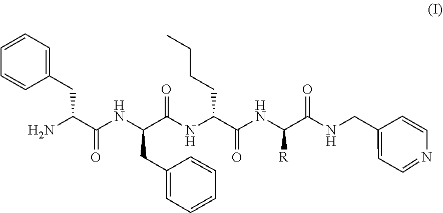

In another embodiment, the sustained release composition includes biocompatible polymeric matrix of ethylene vinyl acetate (EVA) copolymer. in some embodiments, the EVA polymer matrix is a rod shaped implantable device having a diameter of about 0.5 to about 10 mm, and a length of about 0.5 to 10 cm. In some embodiments, the composition includes about 10 to about 85% kappa-opioid agonist of the total weight of the composition.

In another embodiment, a method of treating chronic pain in a subject includes administering to said subject in need thereof a sustained release composition comprising a biocompatible polymeric matrix and a kappa-opioid agonist of formula I, wherein the composition includes a therapeutically effective amount of the kappa-opioid agonist. In some embodiments, the chronic pain is peripheral pain, visceral pain, thermal pain, bone pain, neuropathic pain, chronic low back pain, inflammatory pain, and pain associated with cancer.

The kappa-opioid agonists of formula I exhibit high peripheral to CNS selectivity, and benefit patients with visceral and neuropathic pain. In some embodiments, these kappa-opioid agonists of formula I are highly specific for kappa receptors with little or no agonist or antagonist activity to mu or delta receptors. In some embodiments, the kappa-opioid agonists of formula I do not cause CNS-dependent adverse effects. The kappa-opioid agonists of formula I may not cross blood-brain barrier to elicit side effects.

### BRIEF DESCRIPTION OF THE FIGURES

FIG. 1 illustrates CR665 structure and various analogs of CR665 prepared by modifying the D-Arg residue at position 4 of CR665.

FIG. 2 depicts effect of compounds 1-18 in the acetic acid-induced writhing assay in rats. Results are shown as the mean±SEM with n=3-9 for each compound. Asterisk indicates that the value is significantly different from the saline control value by Student's T-Test, p<0.05.

FIG. 3 shows dose response (and EC₅₀) activation of kappa receptors by compounds #3, 7, 9, 11 and 17 in the DiscoveRx PathHunter™ Beta-Arrestin platform (95% confidence levels (nM); #3:1.38 to 18.67, #7:5.63 to 13.14, #9:15.34 to 54.48, #11:8.15 to 26.77, #17:47.2 to 166.0; CR665:9.07 to 13.14; Dynorphin:10.14 to 96.62). Individual points are the mean±SEM of 3-6 separate repeats.

FIG. 4 shows activation of mu (A) and delta (B) opioid receptors by compounds 3, 7, 9, 11 and 17 in DiscoveRx PathHunter™ Beta-Arrestin platform. Lys-dermorphin and DADLE at 200 nM served as positive controls for mu and delta receptors, respectively. Individual points are the mean±SEM of 3-6 separate repeats. No activation was detected.

FIG. 5 shows antagonism of mu (A) and delta (B) opioid receptors by compounds 3, 7, 9, 11 and 17 in the DiscoveRx PathHunter™ Beta-Arrestin platform. Naloxone served as a positive control antagonist for both mu and delta receptors whereas lys-dermorphin and DADLE at 200 nM were the control agonists, respectively. The mean±SEM of cells given no additions of agonist and/or antagonists is indicated by the x and error bars next to the words “No add”. Individual points are the mean±SEM of 3-6 separate repeats.

FIG. 6 shows oral dose-response evaluation of compound 9 in the acetic acid writhing model (EC50=4.7 mg/kg). Individual points are the mean±SEM, n=3 for all data points.

FIG. 7 shows peripheral selectivity of compound 9. A). i.v dose response in writhing assay (EC50=0.032; 95% confidence level of 0.0095 to 0.1069 mg/kg; Individual points are the mean±SEM, n=3 for all data points). B). hotplate assay of compound 9 at 30 mg/kg. Individual points are the mean±SEM, n=3 for all data points. No change from saline control was detected.

FIG. 8 shows effect of compounds in the acetic acid-induced writhing assay in rats. Results are shown as the mean±SEM with n=2-8 for each compound. Asterisk indicates that the value is not significantly different from the morphine control value by Student's T-Test, *p<0.05.

FIG. 9 shows results of a self-administration procedure. Mean active lever presses and mean infusions (n=8) two-hour session operant self-administration sessions. JT09 failed to maintain lever responding in rats over a five-day period. The number of infusions decreased on all 4 days compared to day one of JT09 administration [F(4,28)=9.04, p<0.0001, and Dunnett post hoc, p<0.05]. Further, to ensure that these rats were not deficient in reward processing, JT09 was replaced with cocaine using a nose-poke operandi and the number of cocaine active lever presses increased over the 7 days [F(6,42)=4.6, p<0.0012] with significantly more active lever presses on days 6 and 7 (Dunnett post hoc, p<0.05).

FIG. 10 shows results of conditioned place preference procedure. JT09 (20 mg/kg, p.o.) had no effect on compartment placement in a conditioned place preference procedure. Baseline preferences for each compartment (black bar) were assessed prior to conditioning and did not change following drug treatment (*p<0.05).

FIG. 11 shows results of a forced swim assay. Immobility time (in seconds) during the last trial of the forced swim test, 30 min after a single dose of JT09 (20 mg/kg, p.o.), salvinorin A (1 mg/kg, i.p.) or saline (2 mL, p.o.). Saline and JT09 were statistically indistinguishable in each trial (Student's t-test, p<0.05). There was a significant interaction between salvinorin A and JT09 [F(3,30)=117, p<0.0001], specifically rats treated with JT09 had lower amounts of time spent immobile relative to salvinorin A during all trials (Sidak's multiple comparison, p<0.05). Further, the main effect of treatment [F(1,10)=947, p<0.0001] and time [F(3,30)=418, p<0.0001] were also significant. Data are expressed as mean±SEM with n=8.

FIG. 12 shows results of a locomotor activity test. Distance Traveled time (in centimeters) during activity tests, 30 min after a single dose of JT09 (20 mg/kg, p.o.), morphine (10 mg/kg, i.p.) or saline (2 mL, p.o.). Saline and JT09 were statistically indistinguishable in all time bins (Student's t-test, p<0.05). There was a significant interaction between morphine and JT09 [F(5,70)=7.0, p<0.0001], specifically JT09 had higher locomotor activity relative to morphine during time bins 1, 2, and 5 (Sidak's multiple comparison, p<0.05). Further, the main effect of treatment [F(1,14)=18.6, p<0.0007] and time [F(5,70)=84, p<0.0001] were also significant. Data are expressed as mean 8.

### DETAILED DESCRIPTION

Pain is the most common symptom that leads people to seek medical intervention in the United States. While estimates of people with chronic pain vary widely, a 2001 study revealed that 50% of individuals reported having ‘any’ chronic pain in the preceding three months, 14% reported having ‘significant’ chronic pain while 6% reported having ‘severe’ chronic pain. These values increase with age, cancer, and in patients that are hospitalized. The great majority of general practitioners (81%) believe that pain management is ineffective in over half of the patients seeking help. The most difficult pain to manage successfully is chronic pain, which includes visceral, thermal, bone, and neuropathic pain, and pain associated with cancer. Currently, there are two major types of chronic pain medications in use—opioids and non-opioids—both of which have important limitations. Non-opioid analgesics include paracetamol and the NSAIDs, all of which target prostaglandin formation, usually through the inhibition of the COX-1 and COX-2 enzymes. Nonselective COX inhibitors result in adverse side-effects associated with COX-1 inhibition, including renal dysfunction, GI ulceration and inhibition of platelet aggregation. Opioids are the main class of analgesics used in the treatment of moderate to severe chronic pain. These compounds have various side effects including nausea, vomiting, constipation, depressed breathing and neurotoxicity. Most significantly, patients can become both addicted and tolerant to these agents, thus requiring dose escalations to maintain therapeutic value.

The mediation of opioid analgesic effects occurs through three receptors—mu, kappa, and delta. Activation of these receptors was long thought to occur only centrally, but in recent years, the receptors have been found in peripheral sensory neurons that can be modulated by endogenous opioids or opioid drugs. Opioids also have anti-inflammatory properties, hence they exhibit more pronounced analgesic effects in damaged (inflammatory) versus normal tissues. This appears to be a result of upregulation of the opioid receptors during inflammatory events and release of opioid peptides (endorphins, enkephalins, dynorphins and others) from immune calls. Mechanical nerve damage leading to neuropathic pain also results in the upregulation of the opioid receptors, and corresponding greater opioid analgesic effects. Endogenous opioid peptides are released in response to damage of stimulatory neurons and by immune cells in response to inflammation, which is consistent with a regulated response to inflammation and attendant pain. Finally, recent studies suggest that systemically and centrally administered opioids may be acting predominantly (50-80%) as agonists of the peripheral opioid receptors.

Opioids differentially target the three opioid receptors, both in the CNS and peripherally, which can lead to untoward side-effects. Agonists for the mu-receptor are the most currently used opioids, but suffer from induction of euphoria, addiction, respiratory depression, and GI tract inhibition. Kappa opioid agonists (KOAs) exhibit none of these effects, and have been shown in visceral pain models to be the most efficacious of the opioids. Potentially more promising are peptidic compounds, including FE20041 and FE200665 (CR665), which exhibit very high peripheral versus central activity and have shown benefit in patients with visceral and neuropathic pain, having the same analgesic effects as the early KOAs without the negative side-effects. However, these peptides are not orally active, which drastically restricts its potential use as a broad spectrum analgesic for peripheral pain.

This disclosure is not limited to the particular systems, devices and methods described, as these may vary. The terminology used in the description is for the purpose of describing the particular versions or embodiments only, and is not intended to limit the scope.

As used herein, the term “alkyl” means a saturated hydrocarbon group which is straight-chained or branched. Alkyl may be heteroalkyl.

As used herein, the term “substituted alkyl” refers to an alkyl as just described in which one or more hydrogen atoms attached to carbon of the alkyl is replaced by another group.

As used herein, the term “heteroalkyl” refers to alkyl groups in which one or more C atoms are replaced by oxygen, nitrogen, sulfur or combinations thereof.

As used herein, the term “alkenyl” means a straight or branched alkyl group having one or more double carbon-carbon bonds. Alkenyl may be heteroalkenyl.

As used herein, the term “substituted alkenyl” refers to an alkenyl as just described in which one or more hydrogen atoms attached to carbon of the alkenyl is replaced by another group.

As used herein, the term “heteroalkenyl” refers to alkenyl groups in which one or more C atoms are replaced by oxygen, nitrogen, sulfur or combinations thereof.

As used herein, the term “alkynyl” means a straight or branched alkyl group having one or more triple carbon-carbon bonds. Alkynyl may be heteroalkynyl.

As used herein, the term “substituted alkynyl” refers to an alkynyl as just described in which one or more hydrogen atoms attached to carbon of the alkynyl is replaced by another group.

As used herein, the term “heteroalkynyl” refers to alkynyl groups in which one or more C atoms are replaced by oxygen, nitrogen, sulfur or combinations thereof.

As used herein, the term “aryl” means a monocyclic, bicyclic, or polycyclic (e.g., having 2, 3 or 4 fused rings) aromatic hydrocarbons. In some embodiments, aryl groups have from 6 to 20 carbon atoms or from 6 to 10 carbon atoms. Examples of aryl groups include, but are not limited to, phenyl, biphenyl, benzyl, naphthyl, anthracenyl, phenanthrenyl, indanyl, indenyl, tetrahydronaphthyl, and the like. Aryl may be heteroaryl.

As used herein, the term “substituted aryl” refers to aryl as just described in which one or more hydrogen atoms attached to any carbon atoms is replaced by one or more functional groups.

As used herein, the term “heteroaryl” means an aromatic heterocycle having up to 20 ring-forming atoms (e.g., C) and having at least one heteroatom ring member (ring-forming atom) such as sulfur, oxygen, or nitrogen. In some embodiments, the heteroaryl group has at least one or more heteroatom ring-forming atoms, each of which are, independently, sulfur, oxygen, or nitrogen.

As used herein, the term “arylalkyl” means a C₁₋₆ alkyl substituted by aryl.

As used herein, the term “heterocyclic ring” means a 5- to 7-membered mono- or bicyclic or 7- to 10-membered bicyclic heterocyclic ring system any ring of which may be saturated or unsaturated, and which consists of carbon atoms and from one to three heteroatoms chosen from N, O and S, and wherein the N and S heteroatoms may optionally be oxidized, and the N heteroatom may optionally be quaternized, and including any bicyclic group in which any of the above-defined heterocyclic rings is fused to a benzene ring.

A “therapeutically effective amount” or “effective amount” of a composition is a predetermined amount calculated to achieve the desired effect, i.e., to supplement, promote, or increase nutritional health. The activity contemplated by the present methods includes both therapeutic and/or prophylactic treatment, as appropriate. The specific dose of a compound administered according to this invention to obtain therapeutic and/or prophylactic effects will, of course, be determined by the particular circumstances surrounding the case, including, for example, the compound administered, the route of administration, and the condition being treated. The effective amount administered may be determined by a physician in the light of the relevant circumstances including the condition to be treated, the choice of compound to be administered, and the chosen route of administration. A therapeutically effective amount of compound of this invention is typically an amount such that when it is administered in a physiologically tolerable excipient composition, it is sufficient to achieve an effective systemic concentration or local concentration in the target tissue.

By “pharmaceutically acceptable”, it is meant the carrier, diluent or excipient must be compatible with the other ingredients of the formulation and not deleterious to the recipient thereof.

As used herein, the term “consists of” or “consisting of” means that the device or method includes only the elements, steps, or ingredients specifically recited in the particular claimed embodiment or claim.

In embodiments or claims where the term comprising is used as the transition phrase, such embodiments can also be envisioned with replacement of the term “comprising” with the terms “consisting of” or “consisting essentially of.”

There exists a critical need for the development of novel compositions for the treatment of pain, which include chronic pain of peripheral origin. Disclosed herein are sustained release compositions of modified CR665. These CR665 analogs contain modifications at position 4 that improve stability and oral availability. CR665 is a D-amino acid containing tetrapeptide (D-Phe-D-Phe-D-Nle-D-Arg-NH-4-Picolyl) having the following structure:

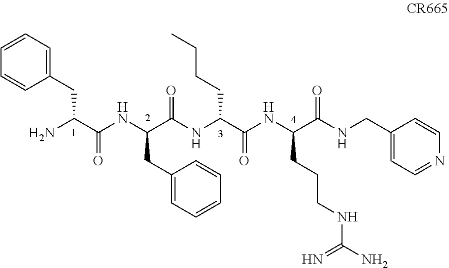 [SMILES: CCCC[C@@H](NC(=O)[C@@H](Cc1ccccc1)NC(=O)[C@H](N)Cc1ccccc1)C(=O)N[C@H](CCCNC(=N)N)C(=O)NCc1ccncc1]

Disclosed herein are analogs of CR665 that contain various structural modifications with opioid agonist activity. The position 4 D-Arg residue of CR665 was converted to derivatives containing the modified D-Arg or D-Lys residues. The modified kappa-opioid agonists disclosed herein exhibit high peripheral to CNS selectivity, and benefit the patients with visceral and neuropathic pain. In some embodiments, these kappa-opioid agonists of formula I are highly specific for kappa receptors with little or no agonist or antagonist activity to mu or delta receptors. In some embodiments, the kappa-opioid agonists of formula I do not cause CNS-dependent adverse effects. The kappa-opioid agonists of formula I may not cross blood-brain barrier to elicit side effects. In some embodiments, the modified analogs of CR65 disclosed herein are part of sustained release compositions. In some embodiments, the modified analogs of CR65 disclosed herein are orally-active compounds.

In some embodiments, the kappa-opioid agonist is of formula I:

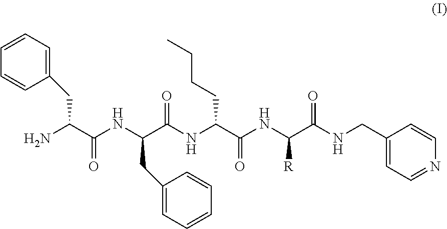

and pharmaceutically acceptable salts, solvates, and stereoisomers thereof.

In some embodiments, R of formula I is represented by formula II or formula III as follows:

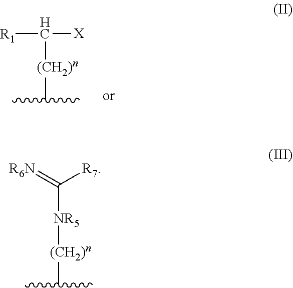

wherein n is an integer from 1 to 4;

X may be —NR₂R₃ or —N^{⊕}R₂R₃R₄;

R₁, R₂, R₃, R₄, are independently, hydrogen, C₁-C₅ alkyl, C₁-C₅ substituted alkyl, C₁-C₅ alkenyl, C₁-C₅ substituted alkenyl, C₁-C₅ alkynyl, C₁-C₅ substituted alkynyl, cycloalkyl, aryl, substituted aryl, or arylalkyl;

R₇ may be hydrogen, C₁-C₅ alkyl, C₁-C₅ substituted alkyl, C₁-C₅ alkenyl, C₁-C₅ substituted alkenyl, C₁-C₅ alkynyl, C₁-C₅ substituted alkynyl, cycloalkyl, aryl, substituted aryl, arylalkyl, or —NR₈R₉;

In some embodiments, R₅, R₆, R₈, R₉, are independently, hydrogen, C₁-C₅ alkyl, C₁-C₅ substituted alkyl, C₁-C₅ alkenyl, C₁-C₅ substituted alkenyl, C₁-C₅ alkynyl, C₁-C₅ substituted alkynyl, cycloalkyl, aryl, substituted aryl, or arylalkyl;

In some embodiments, R₅ and R₉ taken together with the nitrogen atom to which they are attached form a heterocyclic ring;

In some embodiments, R₆ and R₉ taken together with the nitrogen atom to which they are attached form a heterocyclic ring.

Non-limiting embodiments of formula II include:

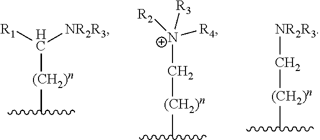

In some embodiments, non-limiting examples of formula III include:

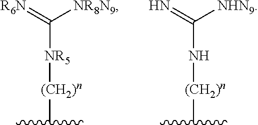

In some embodiments, R₅ and R₉ taken together with the nitrogen atom to which they are attached form a heterocyclic ring as follows:

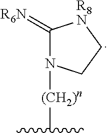

In some embodiments, R₆ and R₉ taken together with the nitrogen atom to which they are attached form a heterocyclic ring as follows:

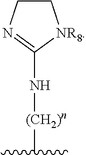

In some embodiments, the stereochemistry at Cα and Cβ C-atoms of the R group in formula I, is independently, either R or S.

Non-limiting embodiments of formula III are:

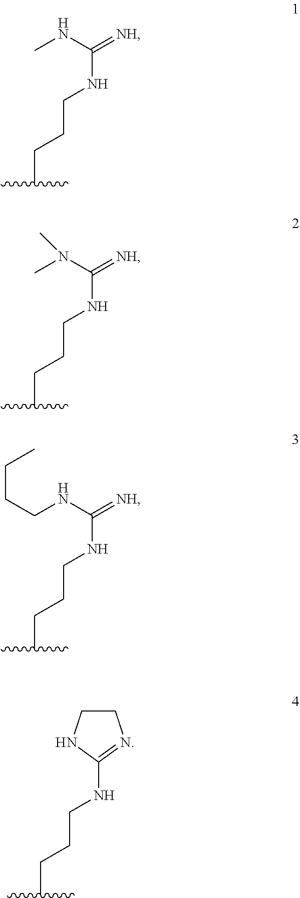

Non-limiting embodiments of formula II are:

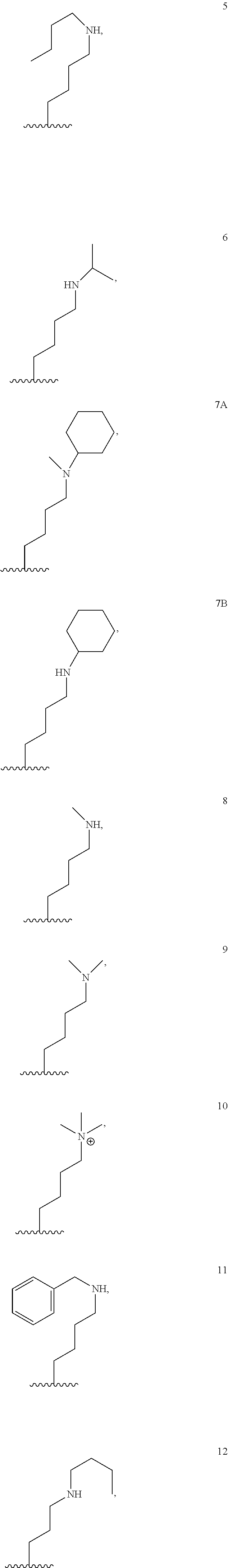

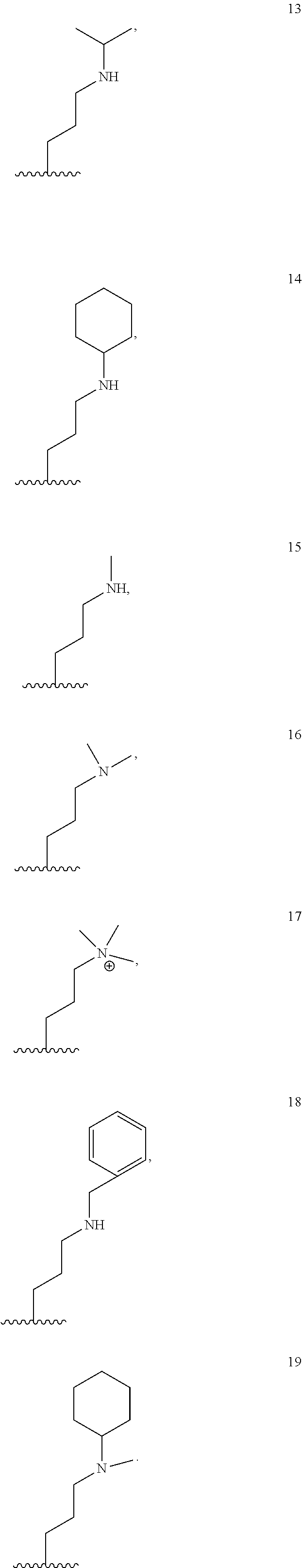

Other mounted D-Arg or D-Lys residues can be substituted at position R of formula I, and are further described in U.S. Pat. Nos. 6,043,218; 6,358,922; 6,566,330; 6,783,946; and 6,858,396, and are incorporated herein by reference.

In some embodiments, kappa-opioid agonist of formula I may have the following substitutions at each of, independently, R₁, R₂, R₃, and R₄, as shown in Table 1.

TABLE 1

| R₁ | R₂ | R₃ | R₄ |
|---|---|---|---|
| hydrogen, C₁—C₅ alkyl, | hydrogen, C₁—C₅ alkyl, | hydrogen, C₁—C₅ alkyl, | |
| C₁—C₅ substituted alkyl, | C₁—C₅ substituted alkyl, | C₁—C₅ substituted alkyl, | |
| C₁—C₅ alkenyl, C₁—C₅ | C₁—C₅ alkenyl, C₁—C₅ | C₁—C₅ alkenyl, C₁—C₅ | |
| substituted alkenyl, C₁—C₅ | substituted alkenyl, C₁—C₅ | substituted alkenyl, C₁—C₅ | |
| alkynyl, C₁—C₅ | alkynyl, C₁—C₅ | alkynyl, C₁—C₅ | |
| substituted alkynyl, | substituted alkynyl, | substituted alkynyl, | |
| cycloalkyl, aryl, | cycloalkyl, aryl, | cycloalkyl, aryl, | |
| substituted aryl, or | substituted aryl, or | substituted aryl, or | |
| arylalkyl. | arylalkyl. | arylalkyl. | |
| hydrogen, C₁—C₅ alkyl, | cycloalkyl, aryl, | cycloalkyl, aryl, | |
| C₁—C₅ alkenyl, C₁—C₅ | substituted aryl, or | substituted aryl, or | |
| alkynyl, cycloalkyl, | arylalkyl. | arylalkyl. | |
| aryl, substituted aryl, or | | | |
| arylalkyl. | | | |
| hydrogen | cycloalkyl, aryl, | cycloalkyl, aryl, | cycloalkyl, aryl, |
| | substituted aryl, or | substituted aryl, or | substituted aryl, or |
| | arylalkyl. | arylalkyl. | arylalkyl. |
| hydrogen | hydrogen, C₁—C₅ alkyl, | hydrogen, C₁—C₅ alkyl, | |
| | C₁—C₅ alkenyl, C₁—C₅ | C₁—C₅ alkenyl, C₁—C₅ | |
| | alkynyl, cycloalkyl, | alkynyl, cycloalkyl, | |
| | aryl, substituted aryl, or | aryl, substituted aryl, or | |
| | arylalkyl. | arylalkyl. | |
| C₁—C₅ alkyl | cycloalkyl, aryl, | cycloalkyl, aryl, | |
| | substituted aryl, or | substituted aryl, or | |
| | arylalkyl. | arylalkyl. | |
| C₁—C₅ alkyl | hydrogen, C₁—C₅ alkyl, | hydrogen, C₁—C₅ alkyl, | |
| | C₁—C₅ alkenyl, C₁—C₅ | C₁—C₅ alkenyl, C₁—C₅ | |
| | alkynyl, cycloalkyl, | alkynyl, cycloalkyl, | |
| | aryl, substituted aryl, or | aryl, substituted aryl, or | |
| | arylalkyl. | arylalkyl. | |
| C₁—C₅ alkyl | hydrogen, C₁—C₅ alkyl, | hydrogen, C₁—C₅ alkyl, | hydrogen, C₁—C₅ alkyl, |
| | C₁—C₅ alkenyl, C₁—C₅ | C₁—C₅ alkenyl, C₁—C₅ | C₁—C₅ alkenyl, C₁—C₅ |
| | alkynyl, cycloalkyl, | alkynyl, cycloalkyl, | alkynyl, cycloalkyl, |
| | aryl, substituted aryl, or | aryl, substituted aryl, or | aryl, substituted aryl, or |
| | arylalkyl. | arylalkyl. | arylalkyl. |

In some embodiments, kappa-opioid agonist of formula I may have the following substitutions at each of, independently, R₅, R₆, R₇, R₈, and R₉, as shown in Table 2.

TABLE 2

| R₅ | R₆ | R₇ | R₈ | R₉ |
|---|---|---|---|---|
| hydrogen, C₁—C₅ | hydrogen, C₁—C₅ | hydrogen, C₁—C₅ | | |
| alkyl, C₁—C₅ | alkyl, C₁—C₅ | alkyl, C₁—C₅ | | |
| substituted | substituted | substituted | | |
| alkyl, C₁—C₅ | alkyl, C₁—C₅ | alkyl, C₁—C₅ | | |
| alkenyl, C₁—C₅ | alkenyl, C₁—C₅ | alkenyl, C₁—C₅ | | |
| substituted | substituted | substituted | | |
| alkenyl, C₁—C₅ | alkenyl, C₁—C₅ | alkenyl, C₁—C₅ | | |
| alkynyl, C₁—C₅ | alkynyl, C₁—C₅ | alkynyl, C₁—C₅ | | |
| substituted | substituted | substituted | | |
| alkynyl, | alkynyl, | alkynyl, | | |
| cycloalkyl, aryl, | cycloalkyl, aryl, | cycloalkyl, aryl, | | |
| substituted aryl, | substituted aryl, | substituted aryl, | | |
| or arylalkyl. | or arylalkyl. | or arylalkyl. | | |
| hydrogen, C₁—C₅ | hydrogen, C₁—C₅ | hydrogen, C₁—C₅ | | |
| alkyl, C₁—C₅ | alkyl, C₁—C₅ | alkyl, C₁—C₅ | | |
| alkenyl, , C₁—C₅ | alkenyl, , C₁—C₅ | alkenyl, , C₁—C₅ | | |
| alkynyl, | alkynyl, | alkynyl, | | |
| cycloalkyl, aryl, | cycloalkyl, aryl, | cycloalkyl, aryl, | | |
| substituted aryl, | substituted aryl, | substituted aryl, | | |
| or arylalkyl. | or arylalkyl. | or arylalkyl. | | |
| hydrogen | hydrogen, C₁—C₅ | hydrogen, C₁—C₅ | | |
| | alkyl, C₁—C₅ | alkyl, C₁—C₅ | | |
| | alkenyl, , C₁—C₅ | alkenyl, , C₁—C₅ | | |
| | alkynyl, | alkynyl, | | |
| | cycloalkyl, aryl, | cycloalkyl, aryl, | | |
| | substituted aryl, | substituted aryl, | | |
| | or arylalkyl. | or arylalkyl. | | |
| hydrogen | cycloalkyl, aryl, | cycloalkyl, aryl, | | |
| | substituted aryl, | substituted aryl, | | |
| | or arylalkyl. | or arylalkyl. | | |
| hydrogen, C₁—C₅ | hydrogen, C₁—C₅ | | hydrogen, C₁—C₅ | hydrogen, C₁—C₅ |
| alkyl, C₁—C₅ | alkyl, C₁—C₅ | | alkyl, C₁—C₅ | alkyl, C₁—C₅ |
| substituted | substituted | | substituted | substituted |
| alkyl, C₁—C₅ | alkyl, C₁—C₅ | | alkyl, C₁—C₅ | alkyl, C₁—C₅ |
| alkenyl, C₁—C₅ | alkenyl, C₁—C₅ | | alkenyl, C₁—C₅ | alkenyl, C₁—C₅ |
| substituted | substituted | | substituted | substituted |
| alkenyl, C₁—C₅ | alkenyl, C₁—C₅ | | alkenyl, C₁—C₅ | alkenyl, C₁—C₅ |
| alkynyl, C₁—C₅ | alkynyl, C₁—C₅ | | alkynyl, C₁—C₅ | alkynyl, C₁—C₅ |
| substituted | substituted | | substituted | substituted |
| alkynyl, | alkynyl, | | alkynyl, | alkynyl, |
| cycloalkyl, aryl, | cycloalkyl, aryl, | | cycloalkyl, aryl, | cycloalkyl, aryl, |
| substituted aryl, | substituted aryl, | | substituted aryl, | substituted aryl, |
| or arylalkyl. | or arylalkyl. | | or arylalkyl. | or arylalkyl. |
| hydrogen, C₁—C₅ | hydrogen, C₁—C₅ | | hydrogen, C₁—C₅ | hydrogen, C₁—C₅ |
| alkyl, C₁—C₅ | alkyl, C₁—C₅ | | alkyl, C₁—C₅ | alkyl, C₁—C₅ |
| alkenyl, , C₁—C₅ | alkenyl, , C₁—C₅ | | alkenyl, , C₁—C₅ | alkenyl, , C₁—C₅ |
| alkynyl, | alkynyl, | | alkynyl, | alkynyl, |
| cycloalkyl, aryl, | cycloalkyl, aryl, | | cycloalkyl, aryl, | cycloalkyl, aryl, |
| substituted aryl, | substituted aryl, | | substituted aryl, | substituted aryl, |
| or arylalkyl. | or arylalkyl. | | or arylalkyl. | or arylalkyl. |
| hydrogen | hydrogen, C₁—C₅ | | hydrogen, C₁—C₅ | hydrogen, C₁—C₅ |
| | alkyl, C₁—C₅ | | alkyl, C₁—C₅ | alkyl, C₁—C₅ |
| | alkenyl, , C₁—C₅ | | alkenyl, , C₁—C₅ | alkenyl, , C₁—C₅ |
| | alkynyl, | | alkynyl, | alkynyl, |
| | cycloalkyl, aryl, | | cycloalkyl, aryl, | cycloalkyl, aryl, |
| | substituted aryl, | | substituted aryl, | substituted aryl, |
| | or arylalkyl. | | or arylalkyl. | or arylalkyl. |
| hydrogen | cycloalkyl, aryl, | | cycloalkyl, aryl, | cycloalkyl, aryl, |
| | substituted aryl, | | substituted aryl, | substituted aryl, |
| | or arylalkyl. | | or arylalkyl. | or arylalkyl. |

### Sustained Release Compositions

Disclosed herein are various sustained release compositions of kappa-opioid agonists for treating peripheral pain. It is to be understood that “kappa-opioid agonists” include kappa-opioid agonists of formula I and other kappa-opioid agonists known in the art unless otherwise indicated. Sustained (or controlled) release refers to the gradual release of kappa-opioid agonist from the composition over a period of time. While there may be an initial burst phase, in some embodiments, it is preferred that the release display relatively linear kinetics, thereby providing a constant supply of the kappa-opioid agonist over the release period. The release period may vary from several hours to several months, depending upon the kappa-opioid agonist and its intended use. It is desirable that the release of the kappa-opioid agonist from the composition over the treatment period be relatively constant. The duration of the release period may be controlled by, inter alia, the composition of the biocompatible polymer matrix, the concentration of the kappa-opioid agonist, the locus of administration, and, addition of release profile modifying agents.

Typically, ethylene vinyl acetate copolymer (EVA) is used as the biocompatible polymeric matrix, but other nonerodible materials may be used. Examples of other suitable materials include silicone, hydrogels such as crosslinked poly(vinyl alcohol) and poly(hydroxy ethylmethacrylate), acyl substituted cellulose acetates and alkyl derivatives thereof, partially and completely hydrolyzed alkylene-vinyl acetate copolymers, unplasticized polyvinyl chloride, crosslinked homo- and copolymers of polyvinyl acetate, crosslinked polyesters of acrylic acid and/or methacrylic acid, polyvinyl alkyl ethers, polyvinyl fluoride, polycarbonate, polyurethane, polyamide, polysulphones, styrene acrylonitrile copolymers, crosslinked poly(ethylene oxide), poly(alkylenes), poly(vinyl imidazole), poly(esters), poly(ethylene terephthalate), polyphosphazenes, and chlorosulphonated polyolefines, and combinations thereof.

Other biocompatible polymeric materials that can be used are poly-lactides (PLA) and poly-glycolides (PGA). They may also include derivatives of PLA or PGA, such as poly butylene succinate (PBS), polyhydroxyalkanoate (PHA), polycaprolactone acid lactone (PCL), polyhydroxybutyrate (PHB), glycolic amyl (PHV), PHB and PHV copolymer (PHBV), and poly lactic acid (PLA)-polyethylene glycol (PEG) copolymers (PLEG).

Sustained release compositions are typically formulated with kappa-opioid agonist loading of about 10% to about 85% by weight of the total composition. For example, the sustained release composition may contain a polymer matrix and about 10% to about 20%, about 20% to about 30%, about 30% to about 40%, about 40% to about 50%, about 50% to about 60%, about 60% to about 70%, about 70% to about 80%, or about 80% to about 85% of kappa-opioid agonist by weight of the total composition. The sustained release compositions may contain one or more kappa-opioid agonists of formula I.

In some embodiments, the ratio of the polymer matrix to the kappa-opioid agonist may be from about 0.001:1 weight % to about 9:1 weight %, about 0.001:1 weight % to about 5:1 weight %, or about 0.001:1 weight % to about 0.05:1 weight %. Specific examples include about 0.001:1 weight %, about 0.005:1 weight %, about 0.01:1 weight %, about 0.05:1 weight %, about 0.1:1 weight %, about 0.5:1 weight %, about 1:1 weight %, about 2:1 weight %, about 3:1 weight %, about 4:1 weight %, about 9:1 weight %, and ranges between any of the two values.

In some embodiments, the sustained release compositions may be implantable (e.g., an implantable device). In some embodiments, the sustained release composition be in the form of tablets, rod-shaped structures, or cylindrical structures, and may be produced using an extrusion process, wherein ground EVA is blended with kappa-opioid agonist, melted, and extruded into rod-shaped structures. Rods are cut into individual implantable devices of the desired length, packaged, and sterilized prior to use. Other methods for encapsulating therapeutic compounds in implantable polymeric, nonerodible sustained release matrices are well known to those of skill in the art. Multiple implantable devices may be used, or the size and shape of the devices may be modified, to achieve a desired overall dosage

In some embodiments, EVA implantable devices are about 0.5 cm to about 10 cm, about 1.5 cm to about 5 cm, about 2 cm to about 6 cm, or about 2 cm to about 3 cm in length. EVA implantable devices are about 0.5 mm to about 10 mm, about 1.5 mm to about 5 mm, or about 2 mm to about 3 mm in diameter.

Once the kappa-opioid agonist of formula I begins to release from the composition, the release process may continue for additional time period (sustained release period). For example, sustained release period may be for about 1 week to about 1 month, about 1 week to about 3 months, about 1 week to about 6 months, about 1 week to about 9 months, about 1 week to about 12 months, about 1 week to about 15 months, about 1 week to about 18 months, or about 1 week to about 24 months. During this sustained release period, drug delivery proceeds at a near constant rate. For example, for a period of about 1 week to 7 weeks, about 2% of drug within the composition may be released. In other embodiments, for a period of about 1 week to 10 weeks, about 3% of drug will be released. In additional embodiments, for a period of about 1 week to 15 weeks, about 4% of drug will be released. In further embodiments, for a period of about 1 week to 26 weeks, about 10% of drug will be released.

In some embodiments, the release rate may also be modified by changing the vinyl acetate content in the EVA polymer matrix. The vinyl acetate content is often about 2% to about 50%, more often about 10% to about 35%, most often about 30% to about 35% by weight of the copolymer. In one embodiment, the vinyl acetate content is about 33% by weight of the copolymer.

The sustained release compositions disclosed herein may further contain a hydrogel. Non-limiting examples of hydrogels include methyl cellulose (MC), ethyl cellulose (EC), ethyl methyl cellulose (EMC), hydroxyethyl cellulose (HEC), hydroxylpropyl cellulose (HPC), hydroxymethyl cellulose (HMC), hydroxypropylmethyl cellulose (HPMC), ethylhydroxyethyl cellulose (EHEC), hydroxyethylmethy cellulose (HEMC), methylhydroxyethyl cellulose (MHEC), methylhydroxypropylcellulose (MHPC), and hydroxyethylcarboxymethyl cellulose (HECMC).

In some embodiments, the sustained release compositions can be in forms which include, but are not limited to, softgels, tablets, capsules, cachets, pellets, pills, powders and granules. Topical dosage forms include, but are not limited to, solutions, powders, fluid emulsions, fluid suspensions, semi-solids, ointments, pastes, creams, gels and jellies, and foams. Parenteral dosage forms include, but are not limited to, solutions, suspensions, emulsions, and dry powder. Formulations can also be in the form of films, pads, wafers, injectables, hydrogels, and the like.

In some embodiments, the sustained release compositions may be in the form of a drug reservoir such as injectable microparticles, passive transdermal/transmucosal drug delivery or electrotransport drug delivery systems. It will be appreciated by those skilled in the art that the inventive formulations described herein can be combined with suitable carriers to prepare alternative drug dosage forms (e.g., oral capsule, topical ointment, rectal and/or vaginal suppositories, buccal patches, or an aerosol spray).

In some embodiments, the sustained release compositions may further comprise pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance. For example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980) can be consulted.

Sustained release compositions disclosed herein can comprise suitable solid or gel phase carriers or excipients. Examples of such carriers or excipients include but are not limited to calcium carbonate, calcium phosphate, various sugars, starches, cellulose derivatives, gelatin, and polymers such as, e.g., polyethylene glycols. In some embodiments, the pharmaceutical excipient may include, without limitation, binders, coating, disintegrants, fillers, diluents, flavors, colors, lubricants, glidants, preservatives, sorbents, sweeteners, conjugated linoleic acid (CLA), gelatin, beeswax, purified water, glycerol, any type of oil, including, without limitation, fish oil or soybean oil, or the like.

In some embodiments, the compositions may include one or more disintegrant component, such as croscarmellose sodium, carmellose calcium, crospovidone, alginic acid, sodium alginate, potassium alginate, calcium alginate, an ion exchange resin, an effervescent system based on food acids and an alkaline carbonate component, clay, talc, starch, pregelatinized starch, sodium starch glycolate, cellulose floc, carboxymethylcellulose, hydroxypropylcellulose, calcium silicate, a metal carbonate, sodium bicarbonate, calcium citrate, or calcium phosphate.

In some embodiments, the compositions may include one or more diluent component, such as mannitol, lactose, sucrose, maltodextrin, sorbitol, xylitol, powdered cellulose, microcrystalline cellulose, carboxymethyl-cellulose, carboxyethylcellulose, methylcellulose, ethylcellulose, hydroxyethylcellulose, methylhydroxyethylcellulose, starch, sodium starch glycolate, pregelatinized starch, a calcium phosphate, a metal carbonate, a metal oxide, or a metal aluminosilicate.

In some embodiments, the compositions may include one or more optional lubricant component, such as stearic acid, metallic stearate, sodium stearyl fumarate, fatty acid, fatty alcohol, fatty acid ester, glyceryl behenate, mineral oil, vegetable oil, paraffin, leucine, silica, silicic acid, talc, propylene glycol fatty acid ester, polyethoxylated castor oil, polyethylene glycol, polypropylene glycol, polyalkylene glycol, polyoxyethylene-glycerol fatty ester, polyoxyethylene fatty alcohol ether, polyethoxylated sterol, polyethoxylated castor oil, polyethoxylated vegetable oil, or sodium chloride.

### Administration

Disclosed herein are methods to treat chronic pain. In one embodiment, a method to treat chronic pain in a subject involves administering a sustained release composition comprising a kappa-opioid agonist of formula I, wherein the sustained release composition releases a therapeutically effective amount of the kappa opioid agonist over a sustained period of time. In some embodiments, the chronic pain may be peripheral pain, visceral pain, thermal, bone, and neuropathic pain, and pain associated with cancer. Other examples of chronic pain include pain related to psychosis, stroke, fibromyalgia, irritable bowel syndrome, chronic arthropathy, inflammatory pain, post herpetic neuralgia, trigeminal neuralgia, migraine, chronic low back pain, refractory angina pectoris (chest pains), interstitial cystitis (inflammation around bladder) and other visceral pains.

In some embodiments, the kappa-opioid agonists of formula I exhibit high peripheral to CNS selectivity, and benefit patients with visceral and neuropathic pain. In some embodiments, the kappa-opioid agonists of formula I are highly specific for kappa receptors with little or no agonist or antagonist activity to mu or delta receptors. In some embodiments, the kappa-opioid agonists of formula I do not cause CNS-dependent adverse effects.

The term “subject” includes animals which can be treated using the methods of the invention. Examples of animals include mammals, such as mice, rabbits, rats, horses, goats, dogs, cats, pigs, cattle, sheep, and primates (e.g. chimpanzees, gorillas, and, preferably, humans).

In some embodiments, one or more anti-inflammatory agents are coadministered along with kappa-opioid agonist of formula I. The anti-inflammatory agent may be encapsulated within the same polymeric matrix as kappa-opioid agonist or in a separate polymeric matrix that does not contain kappa-opioid agonist, or may be administered via a different route, such as orally or via injection, either simultaneously with administration of the kappa-opioid agonist-containing sustained release compositions or at a different time, or on a different schedule such as for example multiple dosing of an oral or injectable formulation. In various embodiments, the anti-inflammatory agent may be a steroid, a NSAID, and/or an antihistamine. In some embodiments, an antioxidant is incorporated into the kappa-opioid agonist-containing polymeric matrix and is coadministered along with kappa-opioid agonist. Examples of agents that may be included in the polymeric matrix are dexamethasone, triamcinolone, betamethasone, clobetasol, cortisone, hydrocortisone, or a pharmaceutically acceptable salt thereof, or a nonsteroidal anti-inflammatory agent (“NSAID”), examples of which include but are not limited to diclofenac potassium diclofenac sodium, diclofenac sodium with misoprostol, diflunisal, etodolac, fenoprofen calcium, flurbiprofen, ibuprofen, indomethacin, ketoprofen, meclofenamate sodium, mefenamic acid, meloxicam, nabumetone, naproxen, naproxen sodium, oxaprozin, piroxicam, sulindac, tolmetin, COX-2 inhibitors (e.g., celecoxib, rofecoxib, valdecoxib), acetylated salicylates (e.g., aspirin), nonacetylated salicylates (e.g., choline, magnesium, and sodium salicylates, salicylate), and/or an antihistamine, examples of which include but are not limited to loratadine (“LT”), astemizole, cetrizine dihydrochloride, chlorpheniramine, dexochlorpheniramine, diphenhydramine, mebhydrolin napadisylate, pheniramine maleate, promethazine, or terfenadine.

In some embodiments, the methods of the invention include administration of another substance in conjunction with administration of kappa-opioid agonist sustained release composition. Such substances include, but are not limited to, levodopa, dopamine agonists, catechol-O-methyltranserase inhibitors, or monoamine oxidase inhibitors, administered orally or intravenously.

In some embodiments, the method includes administering the sustained release composition as an adjuvant therapy. In some embodiments, the method includes administering the sustained release composition as a neo-adjuvant therapy. In some embodiments, the sustained release composition can be administered with other treatments, such as radiation therapy, chemotherapy, targeted therapy, gene therapy, or hormone therapy.

In some embodiments, the sustained release compositions can be used in combination with other agents that are administered systemically. In another embodiment, the sustained release compositions may be administered in combination with one or more anticancer agents which include tamoxifen, toremifen, raloxifene, droloxifene, iodoxyfene, megestrol acetate, anasfrozole, letrazole, borazole, exemestane, flutamide, nilutamide, bicalutamide, cyproterone acetate, goserelin acetate, luprolide, finasteride, herceptin, methotrexate, 5-fluorouracil, cytosine arabinoside, doxorubicin, daunomycin, epirubicin, idarubicin, mitomycin-C, dactinomycin, mithramycin, cisplatin, carboplatin, melphalan, chlorambucil, busulphan, cyclophosphamide, ifosfamide, nitrosoureas, thiotephan, metronicdazole, camptothecin, vincristine, taxol, taxotere, etoposide, teniposide, amsacrine, Irinotecan, topotecan, epothilones, gefitinib, erlotinib, angiogenesis inhibitors, EGF inhibitors, VEGF inhibitors, CDK inhibitors, cytokines, Her1 inhibitors, Her2 inhibitors, and monoclonal antibodies. In another embodiment, the sustained release compositions may be administered in combination with one or more cytokines which include, without limitation, a lymphokine, tumor necrosis factors, tumor necrosis factor-like cytokine, lymphotoxin, interferon, macrophage inflammatory protein, granulocyte monocyte colony stimulating factor, interleukins including, without limitation, interleukin-1, interleukin-2, interleukin-6, interleukin-12, interleukin-15, and interleukin-18.

Specific modes of administration will depend on the indication. The selection of the specific route of administration and the dose regimen is to be adjusted or titrated by the clinician according to methods known to the clinician in order to obtain the optimal clinical response. The amount of compounds to be administered is that amount which is therapeutically effective. The dosage to be administered will depend on the characteristics of the subject being treated, e.g., the particular animal or human being treated, age, weight, health, types of concurrent treatment, if any, and frequency of treatments, and can be easily determined by one of skill in the art (e.g., by the clinician).

The dimensions of the sustained release compositions commensurate with the size and shape of the region selected as the site of administration and will not migrate from the insertion site following implantation, injection or other means of depot administration. The sustained release compositions may be rigid, or somewhat flexible so as to facilitate both insertion of the implant at the target site and accommodation of the implant. The sustained release compositions may be particles, sheets, patches, plaques, fibers, microcapsules and the like and may be of any size or shape compatible with the selected site of insertion.

In one embodiment, the sustained release composition may be administered by a catheter. In another embodiment, the sustained release composition may be administered by a syringe. The sustained release composition is formulated so that the composition can be readily implanted (e.g., by injection) into the desired location to form a mass that can remain in place for the period suitable for controlled release of the kappa-opioid agonist and for any additional benefit of mechanical support if applicable. The mechanical and rheological properties suitable for injectable depot compositions are known in the art. Typically, the polymer of the depot vehicle with particulates are present in an appropriate amount of solvent such that the depot composition can be so implanted.

An alternative embodiment of the invention provides for a rod depot implant. Other embodiments include a drug depot implant comprising a hollow depot, the hollow depot comprising a therapeutic agent that provides a concentration gradient for targeted delivery of the agent to the synovial joint, the disc space, the spinal canal, or the soft tissue surrounding the spinal canal of a subject.

In various embodiments of the invention, the sustained release composition is directly administered to the area of the chronic pain by, for example, local infusion during surgery, topical application (e.g., in conjunction with a wound dressing after surgery), injection, means of a catheter, means of a suppository, or means of an implant. An implant can be of a porous, non-porous, or gelatinous material, including membranes, such as sialastic membranes, or fibers. Suppositories generally contain active ingredients in the range of 0.5% to 10% by weight.

In other embodiments, a controlled release system can be placed in proximity of the pain. For example, a micropump may deliver controlled doses directly into the area of the pain, thereby finely regulating the timing and concentration of the pharmaceutical composition.

In some embodiments, the kappa-opioid agonists disclosed herein may not be part of sustained release compositions. The kappa-opioid agonists may be in compositions that can be administered systemic, parenteral, topical, or oral. For example, administration can be, but is not limited to, parenteral, subcutaneous, intravenous, intramuscular, intraperitoneal, transdermal, oral, buccal, ocular routes, or intravaginally, by inhalation, by depot injections, or by implants. In other embodiments, administration can be at the site of tumor resection. Thus, modes of administration of the composition of the present invention (either alone or in combination with other pharmaceuticals) can be, but are not limited to, sublingual, injectable (including short-acting, depot, implant and pellet forms injected subcutaneously or intramuscularly), or by use of vaginal creams, suppositories, pessaries, vaginal rings, rectal suppositories, intrauterine devices, and transdermal forms such as patches and creams.

For oral administration, the pharmaceutical composition can be formulated as tablets, pills, dragees, capsules, liquids, gels, syrups, slurries, suspensions and the like, for oral ingestion by a patient to be treated. Pharmaceutical preparations for oral use can be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinyl-pyrrolidone (PVP). If desired, disintegrating agents can be added, such as, but not limited to, the cross-linked polyvinyl pyrrolidone, agar, or alginic acid or a salt thereof such as sodium alginate.

For oral administration, the hydrogel formulation is preferably encapsulated by a retardant coating, e.g., a bioerodible polymer. Upon dissolution or erosion of the encapsulating material, the hydrogel core becomes exposed and the drug contained within the gel can be released for enteric adsorption. Bioerodible coating materials may be selected from a variety of natural and synthetic polymers, depending on the agent to be coated and the desired release characteristics. Exemplary coating materials include gelatins, carnauba wax, shellacs, ethylcellulose, cellulose acetate phthalate or cellulose acetate butyrate. Release of the agent is controlled by adjusting the thickness and dissolution rate of the polymeric coat.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as, e.g., lactose, binders such as, e.g., starches, and/or lubricants such as, e.g., talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the compositions can take the form of, e.g., tablets or lozenges formulated in a conventional manner.

For administration by inhalation, the compositions for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The compositions of the present invention can also be formulated in rectal compositions such as suppositories or retention enemas, e.g., containing conventional suppository bases such as cocoa butter or other glycerides.

In addition to the formulations described previously, the compositions of the present invention can also be formulated as a depot preparation. Such long acting formulations can be administered by implantation (for example subcutaneously or intramuscularly) or by intramuscular injection.

In transdermal administration, the compositions of the present invention, for example, can be applied to a plaster, or can be applied by transdermal, therapeutic systems that are consequently supplied to the organism.

### Packs and Kits

The sustained release compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

The invention also provides kits for carrying out the therapeutic regimens of the invention. Such kits comprise in one or more containers having therapeutically or prophylactically effective amounts of the sustained release compositions in pharmaceutically acceptable form. The sustained release compositions in a vial of a kit of the invention may be in the form of a pharmaceutically acceptable solution, e.g., in combination with sterile saline, dextrose solution, or buffered solution, or other pharmaceutically acceptable sterile fluid. Alternatively, the complex may be lyophilized or desiccated; in this instance, the kit optionally further comprises in a container a pharmaceutically acceptable solution (e.g., saline, dextrose solution, etc.), preferably sterile, to reconstitute the complex to form a solution for injection purposes.

In another embodiment, a kit of the invention further comprises a needle or syringe, preferably packaged in sterile form, for injecting the complex, and/or a packaged alcohol pad. Instructions are optionally included for administration of sustained release compositions by a clinician or by the patient.

In another embodiment, the kit may contain at least one implantable, nonerodible device of the type herein described, capable of delivering long-term therapeutic levels of kappa-opioid agonist, in suitable packaging, along with instructions providing information to the user and/or health care provider regarding subcutaneous implantation and use of the system for treating a condition for which kappa-opioid agonist administration is therapeutically beneficial.

### Dosage

In some embodiments, about 1 microgram to 500 grams of the sustained release composition is administered. In some embodiments, about 1 microgram to 400 grams of the sustained release composition is administered. In some embodiments, about 1 microgram to 300 grams of the sustained release composition is administered. In some embodiments, about 1 microgram to 200 grams of the sustained release composition is administered.

In one embodiment, the effective dose of the kappa-opioid agonist that is released from the sustained release compositions may range from about 0.1 to 3000, 0.2 to 900, 0.3 to 800, 0.4 to 700, 0.5 to 600, 0.6 to 500, 70 to 400, 80 to 300, 90 to 200, or 100 to 150 micrograms/day. In other embodiments, the dose may range from approximately 10 to 20, 21 to 40, 41 to 80, 81 to 100, 101 to 130, 131 to 150, 151 to 200, 201 to 280, 281 to 350, 351 to 500, 501 to 1000, 1001 to 2000, or 2001 to 3000 nanograms/day. In specific embodiments, the dose may be at least approximately 20, 40, 80, 130, 200, 280, 400, 500, 750, 1000, 2000, or 3000 micrograms/dose. In specific embodiments, the dose may be at least approximately 20, 40, 80, 130, 200, 280, 400, 500, 750, 1000, 2000, or 3000 nanograms/dose.

In another embodiment, the effective dose of the kappa-opioid agonist that is released results in a plasma concentration of approximately 0.1, 1, 2.5, 5, 7.5, 10, 15, 20, 30, 40, or 50 micrograms/liter. In another embodiment, the effective dose of the kappa-opioid agonist that is released results in a plasma concentration of approximately 0.1, 1, 2.5, 5, 7.5, 10, 15, 20, 30, 40, or 50 nanograms/liter. In other embodiments, the resulting circulating concentration of the kappa-opioid agonist is approximately 0.1 to 50, 1 to 40, 2.5 to 30, 5 to 20, or 7.5 to 10 micrograms/liter. In other embodiments, the resulting circulating concentration of the kappa-opioid agonist is approximately 0.1 to 50, 1 to 40, 2.5 to 30, 5 to 20, or 7.5 to 10 nanograms/liter. In other embodiments, the resulting circulating concentration of the kappa-opioid agonist is approximately 0.1 to 1, 1.1 to 2.4, 2.5 to 5, 5.1 to 7.4, 7.5 to 10, 11 to 15, 16 to 20, 21 to 30, 31 to 40, or 41 to 50 micrograms/liter. In other embodiments, the resulting circulating concentration of the kappa-opioid agonist is approximately 0.1 to 1, 1.1 to 2.4, 2.5 to 5, 5.1 to 7.4, 7.5 to 10, 11 to 15, 16 to 20, 21 to 30, 31 to 40, or 41 to 50 nanograms/liter.

In some embodiments, an implantable device of the invention may release kappa-opioid agonist of formula I in vitro or in vivo at a rate of about 0.01 to about 10 mg/day, about 0.1 to about 10 mg/day, about 0.25 to about 5 mg/day, or about 1 to about 3 mg/day in vitro or in vivo. In some embodiments, an implantable device of the invention may release kappa-opioid agonist continuously in vivo at a rate that results in a plasma level of at least about 0.001, 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1,5, or 10 ng/ml of the agonist in the subject.

### EXAMPLES

### Example 1: Synthesis of Kappa Opioid Agonists

Methods

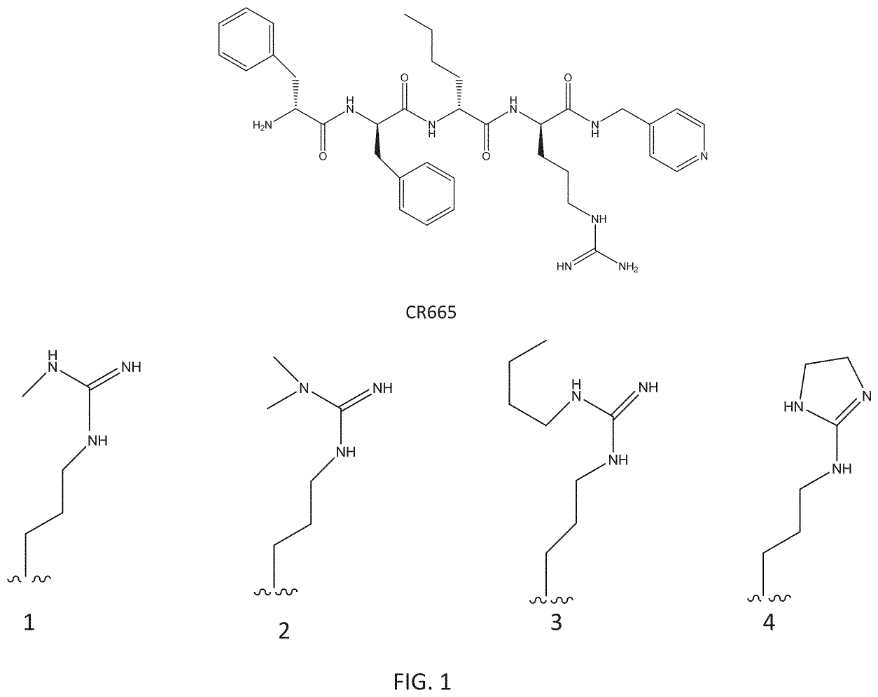

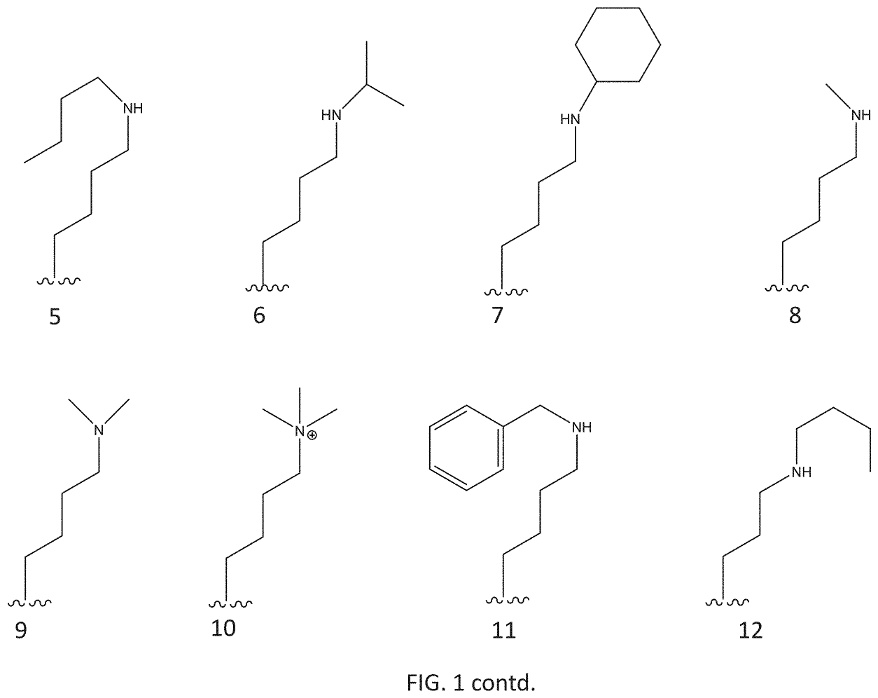

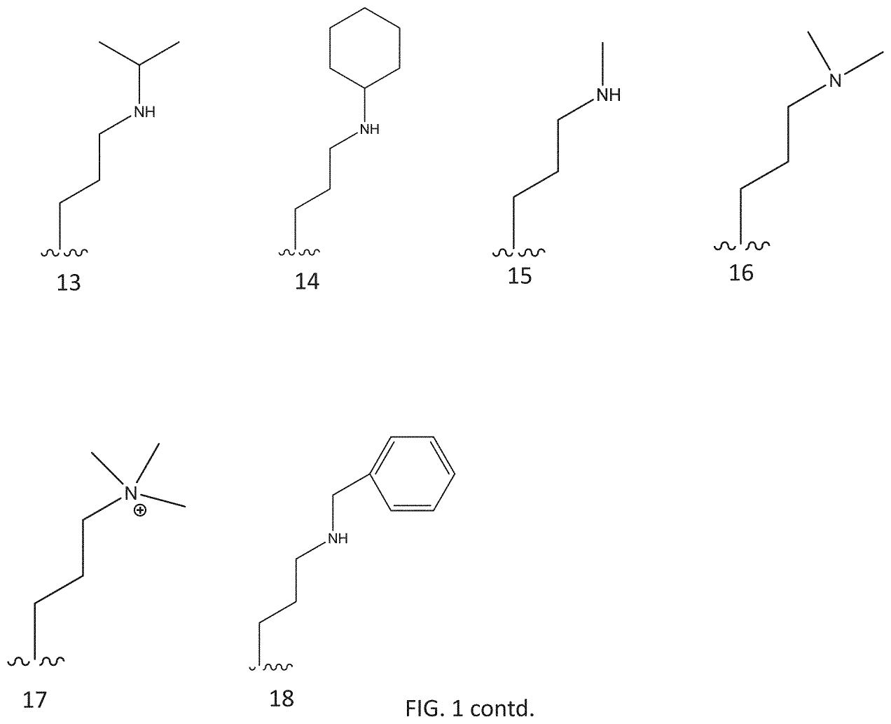

The position 4 D-Arg residue of CR665 was converted to derivatives containing the modified D-Arg or D-Lys residues shown in (FIG. 1). Compounds 1-4 feature alkylations of the D-Arg residue whereas compounds 5-18 feature modifications to the side-chain length and alkylation of D-Lys residue. All compounds were assembled using standard Merrifield chemistry to build the peptides while incorporating the non-natural D-Arg and D-Lys residues, and synthesized. All compounds were HPLC-purified and characterized by MALDI-mass spectrometry, giving measured [M+1]⁺ molecular weights within 0.1% of theoretical values.

Animals.

All animal work was reviewed and approved by the Institutional Animal Care and Use Committee (IACUC) at the Medical University of South Carolina. All experimental protocols were performed in accordance with the guidelines set forth in the NIH Guide for the Care and Use of Laboratory Animals, published by the Public Health Service. All protocols were performed on male Sprague-Dawley rats (Harlan, Prattville Ala., 240-280 g), which were housed in an AAALAC-approved colony room maintained at a constant temperature and humidity. Animals (two per cage; approximately 175-225 g each) were kept on a 12-h light:dark cycle with ad libitum access to food and water. Because many of the assays are susceptible to learning phenomena, which results in progressive decrease of the physiological response or behavior to be monitored, rats were used no more than three times per analgesic assay.

Acetic Acid-Induced Writhing Model.

This protocol is a well-established model of peripheral pain. Following oral or i.v. administration of compound, rats are rested for 20 min before i.p. injection with 2 ml/kg of 3% acetic acid. Animals are then rested an additional 10 min before being placed in a 10×10 inch chamber. A video of the rats is then recorded for next 20 min. The video is subsequently blindly scored by an investigator who examines each rat every 20 sec for the entire 20 min (60 individual observations). At each observation rats are scored on whether they are writhing or not. Writhing is defined as a constriction of the abdominal area, often with extension of the hind legs. At the end of the experiment the percent of the time the animal was writhing was calculated. The dose-response curves and EC50 values were generated using GraphPad Prism®.

Hotplate Model.

The hotplate model evaluates central pain attenuation in a rodent after applying an acute thermal stimuli. The rat is treated with compound and assessed over time on a hotplate analgesia meter (Columbus Instruments, Columbus, Ohio), essentially a flat surface maintained at 53.0+/−0.2° C. The time until the rat lifts, nibbles or shakes one of its hind paws is recorded, which is known as the response latency. Animals remain on the hotplate no longer than 30 sec to avoid tissue damage. Experiments are scored as the percent of maximal possible effect (% MPE) calculated using the following equation: % MPE=[(post-drug latency−pre-drug latency)/(cut-off pre-drug latency)]×100%. The dose response curves and EC50 values were generated using GraphPad Prism®.

Opioid Receptor Activation Assay.

PathHunter™ Beta-Arrestin GPCR Functional Profiling and Screening cell lines that express the individual opioid receptors were obtained from DiscoveRx (Fremont, Calif.). These cells express the kappa (or mu or delta) opioid receptor fused to a proprietary β-galactosidase enzyme fragment and the □-arrestin gene fused to an enzyme acceptor (the remaining fragment of β-galactosidase). Upon activation of the receptor, β-arrestin is recruited to the GPCR and the β-galactosidase is activated through enzyme complementation. Cells were grown using standard cell culture techniques and then plated at 30,000 cells/well in 90 μl in a 96 well white, clear bottom plate and incubated overnight. Dilutions were prepared in dilution buffer (HBBS, 20 mMHepes, 0.1% BSA) and 10 μl added per well. Plates were then incubated for 90 min at 37° C. in a water-saturated atmosphere. Plates were then allowed to come to room temp and DiscoveRx's proprietary developing agent added as recommended by the manufacturer. This agent contains a detergent, 6-O-β-galactopyranosyl-luciferen and luciferase. Plates were then incubated for 1 hr at room temperature before being read on a luminometer. Antagonist studies were performed in an identical manner except after the overnight incubation 10 μl media was removed and replaced with drug or naloxone at the indicated concentration. Cells were then incubated for 30 min at 37° C. in a water-saturated atmosphere before addition of 10 μl of the indicated agonist (lys-dermorphin for mu receptors and [D-Ala2, D-Leu5]-enkephalin (DADLE) for delta receptors). All plates had wells treated with dilution buffer only (background) and others treated with a concentration of agonist shown in preliminary studies to be maximal (200 nM; maximal luminescence). To calculate % maximal luminescence, background was subtracted from the experimental values and this was divided by maximal luminescence and multiplied by 100.

Statistics.

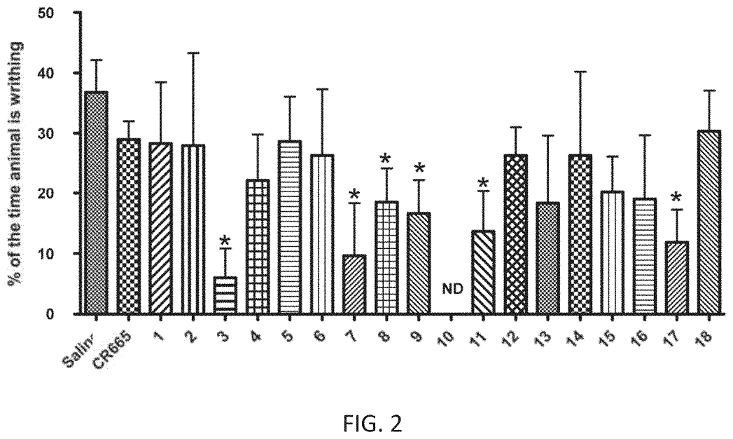

To test for significant differences in the writing assay during the screening of doses (FIG. 2) a Student's t-test was used comparing each compound individually to saline control. Results were considered significantly different if p<0.05. For all dose response experiments (opioid receptor binding assays and writhing dose response) EC50s and 95% confidence limits were calculated using Graphpad Prism software.

Results

FIG. (1) illustrates the series of KOA peptide derivatives that were synthesized and evaluated. All feature modifications to the Position 4 D-Arg residue, and were chosen to provide a range of structures to initially probe potential structure-activity relationships (SARs) at this position. Compounds 1-4 are modified D-Args, compounds 5-11 are modified D-Lys while compounds 12-18 are D-Orns (or 5-11 truncated by one methylene group in the side-chain).

Screening of Potential KOAs.

As an initial biological screen the potential for oral availability of the 18 peptides in our library were evaluated in a well-established model of peripheral pain, the acetic acid-induced writhing assay. Rats were administered the various compounds by oral gavage at a screening dose of 20 mg/kg 20 min before i.p. injection with 2 ml/kg of 3% acetic acid. 10 min later animals were assessed for writhing as indicated in the methods section. As shown in (FIG. 2), several of the compounds (3, 7, 8, 9, 11 and 17) were able to significantly block outward physical signs of peripheral pain in this assay (writhing) while the parent compound, CR665, did not have a significant effect. Thus, several of the Arg modifications can impart CR665 with the ability to cross the gut barrier. Compounds 3, 7, 9, 11 and 17 were selected for further analysis.

Analysis of Kappa Receptor Activation by the KOA Compounds.

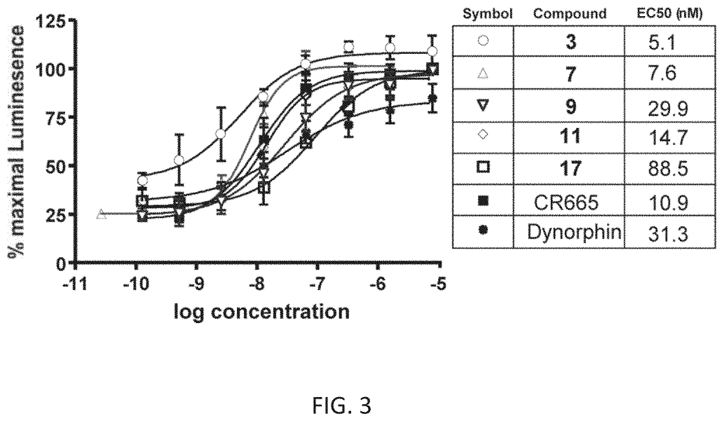

The current compounds, like CR665, are intended to specifically activate the kappa opioid receptor. To confirm this, and to determine their relative activities, we assessed their efficiency using PathHunter™ Beta-Arrestin GPCR Functional Profiling and Screening cell lines from DiscoveRx (Fremont, Calif.) as described in the Materials and Methods section. As shown in (FIG. 3), compounds 3, 7, 9, 11 and 17 activate the kappa opioid receptor with EC50s in the low nM range, as did CR665 and dynorphin (the positive control).

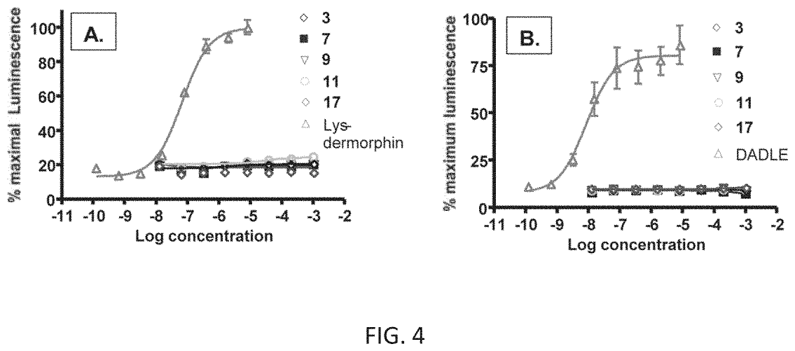

For a peripheral kappa agonist to be effective with minimal side effects it is important that it not interact to any substantial degree with other opioid receptors. Thus, we have used the cloned human receptors in the DiscoveRx system to assess activation at mu or delta receptors. As shown in (FIG. 4), none of our compounds were able to activate the mu (FIG. 4A) or delta (4B) opioid receptors, even at mM concentrations. This indicates a kappa specificity for these compounds from >11,000 fold for 17 to >33,400 for 9 to >200,000 fold for 3. These are minimal estimates; since there were no detectable mu or delta activity, the actual selectivities may be much higher. Lys-dermorphn and DADLE are included as positive controls and have EC50s in the nM range.

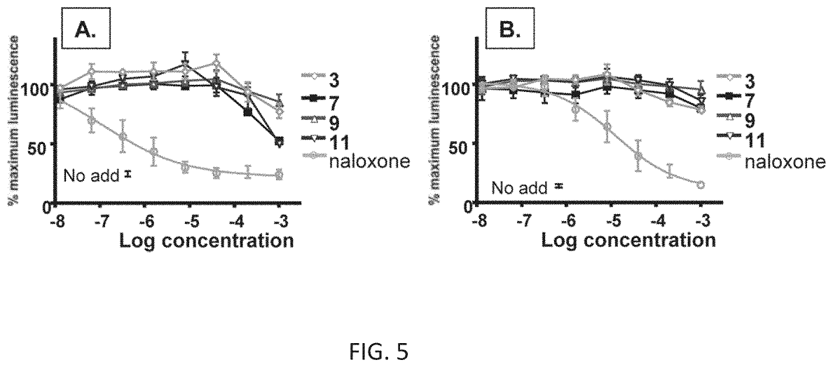

To insure the compounds did not act as antagonists at the non-kappa opioid receptors, cells of the DiscoveRx system that express mu receptors exclusively were first incubated with compound (or the non-selective opioid antagonist naloxone as a positive control) followed by agonist (Lysdermorphin or DADLE at 200 nM). As shown in (FIG. 5A), only slight antagonist effects on mu receptors were detected and then only at the 1 mM concentration. Likewise, similar studies were performed using DiscoveRx cells that exclusively express delta receptors. None of the tested compounds exerted any significant antagonism with these receptors (FIG. 5B). The effects of the naloxone in these studies were similar to published studies.

Oral Dose Response and Peripheral Selectivity of Compound 9.

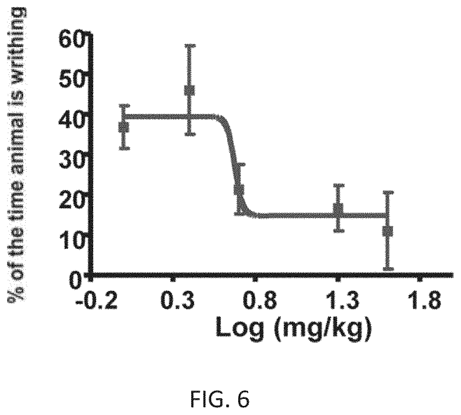

Five compounds, 3, 7, 9, 11 and 17 exhibited extensive yet statistically indistinguishable activity in the various in vitro and in vivo experimental evaluations (FIGS. 2-6). Compound 9 was chosen for proof of concept studies to evaluate potential oral efficacy and peripheral selectivity, based on the ease of synthesis of its particular non-natural Lys residue compared to the other candidates. As shown in (FIG. 6), the analgesic effect of compound 9 in the writhing model of peripheral pain when administered orally is doseresponsive with an EC50 of 4.7 mg/kg.

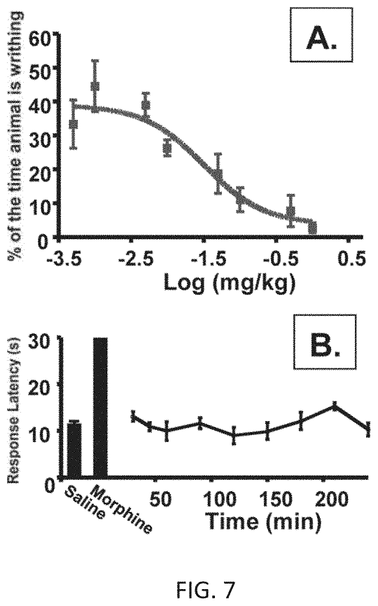

As discussed, central availability of a kappa agonist can lead to undesirable side effects. To assess the peripheral to central selectivity of compound 9 an i.v. dose response of this drug was performed in both a model of peripheral pain (A. the writhing assay) and central pain (B. the hotplate assay). The ratio of EC50s for each of these assays can then be used to determine the peripheral selectivity of the drug. Administration via the i.v. route was used to insure the results do not reflect the ability of compound 9 to cross the gut barrier but rather describe the effective partitioning of the drug once it is in the bloodstream. As shown in (FIG. 7A), compound 9 displayed a dose-response in the writhing assay with a markedly potent EC50 of 0.032 mg/kg. In contrast, no activity could be detected in the hotplate assay with the highest dose of compound 9 tested (30 mg/kg; FIG. 7B). For comparison, morphine at 5 mg/kg will elicit a maximum response after 15 min (shown on the graph), which wanes during the course to the experiment. Using 30 mg/kg as a minimum EC50 in CNS-mediated pain, we can calculate a peripheral selectivity for compound 9 of >900-fold.

### Example 2: Pre-Clinical Studies of Kappa-Opioid Agonists

Methods

Acetic Acid-Induced Writhing Model.

This protocol is a well-established model of peripheral pain. Experimental rats were orally gavaged with each test compound (20 mg/kg) and rested for 20 min before receiving an i.p. (intraperitoneal) injection with 2 ml/kg of 3% acetic acid. Control rats were given an i.p. injection of morphine (10 mg/kg) and rested for 20 min before receiving an i.p. injection with 2 ml/kg of 3% acetic acid. After receiving acetic acid injections, animals were rested for an additional 10 min before being placed in a 10×10 inch Plexiglas chamber. A video of each rat was then recorded for next 20 min. The video was subsequently scored blindly by an investigator who examined each rat every 20 sec for the entire 20 min (60 individual observations). At each observation, rats were scored on whether they are writhing or not. Writhing is defined as a constriction of the abdominal area, often with extension of the hind legs. At the end of the experiment, the percent of time the animal was writhing was calculated. The dose-response curves and EC50 values were generated using GraphPad Prism.

Hot Plate Model.

The hotplate model evaluates central pain attenuation in a rodent after applying an acute thermal stimuli. Experimental rats were orally gavaged with lead compounds JT07 or JT09 (20 mg/kg) and rested for 20 min prior to being placed on the hotplate. Control rats received i.p. injections of morphine (10 mg/kg) and rested for 20 min prior to be being placed on the hotplate. Rats were then assessed over time on a hotplate analgesia meter (Columbus Instruments, Columbus, Ohio), essentially a flat surface maintained at 53.0+/−0.2° C. The time until the rat lifted, nibbled, or shook one of its hind paws was recorded, which is known as the response latency. Animals remained on the hotplate no longer that 30 sec to avoid tissue damage. Experiments were scored as the percent of maximal possible effect (% MPE) calculated using the following equation: % MPE=[(post-drug latency−pre-drug latency)/(cut-off pre-drug latency)]×100%. The dose response curves and EC50 values were generated using GraphPad Prism.

Self-Administration Model.

Addiction/dependence is a critical side effect of opioids and must be evaluated with any new member of this family. The most reliable test of abuse liability with the most translational relevance is a contingent drug self-administration model, in which rats are trained to press a lever for intravenous drug delivery. Sixteen standard self-administration chambers (30×20×20 cm, Med Associates) were housed inside sound-attenuating cubicles fitted with a fan for airflow and to mask noise. Each chamber contained two retractable levers, two stimulus lights, a speaker for tone delivery, and a house light to provide general illumination. In addition, each chamber was equipped with a balanced metal arm and spring leash attached to a swivel (Instech). Tygon® tubing extended through the leash and was connected to a 10 ml syringe mounted on an infusion pump located outside the sound-attenuating cubicle. Rats were anesthetized with i.p. injections of ketamine (66 mg/kg; Vedcolnc, St Joseph, Mo., USA), xylazine (1.3 mg/kg; Lloyd Laboratories, Shenandoah, Iowa, USA), and equithesin (0.5 ml/kg); sodium pentobarbital (4 mg/kg), chloral hydrate (17 mg/kg), and 21.3 mg/kg magnesium sulfate heptahydrate dissolved in 44% propylene glycol, 10% ethanol solution). Ketorolac (2.0 mg/kg, i.p. Sigma, St. Louis, Mo., USA) was given just prior to surgery as an analgesic. One end of a silastic catheter was inserted 33 mm into the external right jugular and secured with 4.0 silk sutures. The other end ran subcutaneously and exited from a small incision just below the scapula. This end is attached to an infusion harness (Instech Solomon, Plymouth Meeting, Pa., USA) that provides access to an external port for IV drug delivery. Following this surgical procedure, rats were given a subcutaneous injection of antibiotic solution of Cefazolin (10 mg/0.1 ml; Schein Pharmaceuticals, Florham Park, N.J., USA) and allowed to recover for 5 days.

During self-administration, rats received an IV infusion (0.1 ml) of 10 U/ml heparinized saline before each session. After each session, catheters were flushed with cefazolin and 0.1 ml 70 U/ml heparinized saline. Catheter patency was periodically verified with methohexital sodium (10 mg/ml dissolved in 0.9% physiological saline), a short-acting barbiturate that produces a rapid loss of muscle tone when administered IV. Daily 2-hr sessions occurred in a fixed ratio 1 schedule of reinforcement. The house light signaled the beginning of a session and remained on throughout the session. During the sessions, a response on the active lever resulted in activation of the pump for a 2-s infusion (50 bolus infusion) and presentation of a stimulus complex consisting of a 5-s tone (78 dB, 4.5 kHz) and a white stimulus light over the active lever, followed by a 20-s time out. Responses occurring during the time out and on the inactive lever were recorded, but had no scheduled consequences. Rats were trained initially to press a lever for a sucrose pellet (45 mg) in a single 6 hr session. The following day, a response on the lever no longer resulted in sucrose reward, but instead produced an IV infusion of JT09 (20 mg/kg/inf). After five days, JT09 was replaced with cocaine (50 ug/50 ul bolus infusion) and the lever replaced with a nose poke aperture. Cocaine was chosen for comparison purposes because it is readily self-administered.

Conditioned Place Preference.

To test whether JT09 was able to condition appetitive behaviors, we used a conditioned place procedure. On day one, rats were habituated to a three-compartment apparatus for 10 min. One compartment was black with a grid floor and the other white with a rod floor. A smaller center compartment was gray with a solid floor. The amount of time spent in each compartment was recorded. The side in which the rats spent the least amount of time on habituation day was paired with JT09, in that rats receive the compound via oral gavage immediately before compartment placement. On alternating days, rats were treated with saline and confined in the opposite compartment. Rats were confined to each compartment for 25 minutes. Test rats were orally gavaged with saline and given access to the entire apparatus for 10 minutes. The amount of time spent in each compartment was recorded.

Forced Swim Assay.

The most common reason for discontinuing development of kappa agonists is the induction of dysphoria, which is mediated through CNS kappa receptors. To test for this side effect, we used an established model: the repeated forced swim assay. Rats were placed in a container with 30° C. water without an escape avenue. Time spent immobile during the last 4 min of each trial was recorded. Immobility was defined by a posture in which the forelimbs were motionless in front of the body, hind legs display limited motion, and the tail is directed outward. On day one, rats were placed in the water for 15 min. On the following day, placements consisted of four trials lasting 6 min each, separated by 10 mins. Salvorin A (1 mg/kg, i.p.) or JT09 (20 mg/kg, p.o.) were administered immediately before the first placement. Time spent immobile is a diagnostic of dysphoria and typically increases with each trial. Salvinorin A (a centrally available kappa agonist) was used as a positive control due to its known anti-depressant-like effects such as dysphoria.

Locomotor Open Field Test.

Sedation and a loss of muscle coordination are common CNS-mediated side effects of opioids. To assess the sedative effects of JT09, rats were given JT09 (20 mg/kg, p.o.) or morphine (10 mg/kg, i.p.) 20 min before placement in an automated (68 l×21 w×21 h cm) activity chambers for a 30 min test. Morphine was used as a comparison group due to its well-known sedative effects.

Maximum Tolerated Dose Determination.

In order to determine the maximum dose of JT09 that can be tolerated, its dose level was increased until the maximum tolerated dose (MTD) was identified or, if MTD dose was not reached, the maximum-administered dose (MAD). The MTD is a dose that does not produce mortality, a loss of more than 10% body weight, or overt signs of toxicity. Six rats were included at each dose level (50, 70, and 90 mg/kg, p.o.). The animals were observed twice daily; body weights and detailed clinical observations for behavioral toxicity (porphyrin staining, changes in activity levels, changes in grooming habits, convulsions, catalepsy, muscular rigidity, and excessive vocalization) were made daily for 4 days after dosing at each level. Doses were chosen based on the compound's ED₅₀ for analgesic potency.

Multiple Dose Study.

Rats were be orally treated with JT09 daily at its ED90 (30 mg/kg) for 14 consecutive days. Tolerance to the analgesic effects of the lead compounds was be assessed using the acetic acid-induced writhing assay (described above). Daily clinical observations for behavioral toxicity (described above) were made during the course of the experiment and body weight and food consumption recorded on study days 3, 5, 7, 9, 11 and 13. On day 15, animals were sacrificed in their home cages by carbon dioxide inhalation from a compressed gas tank. A gross necroscopic analysis was immediately performed following euthanasia.

Statistics.

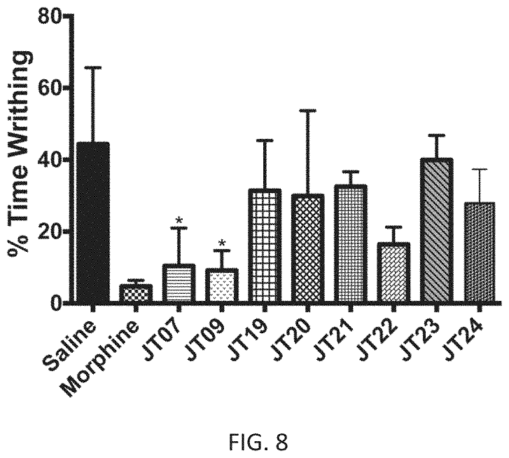

To test for significant differences in the acetic acid-induced rat writhing assays and hot plate assays (FIG. 8) a Student's t-test was used comparing each compound individually to the control groups. Results were considered significantly different if p<0.05. For all dose response experiments (writhing dose response assays) EC50s and 95% confidence limits were calculated using Graphpad Prism software. CPP was analyzed with a Student's t-test to compare the amount of time rats spent in each compartment. Self-administration, locomotor, and forced swim data were analyzed by two-way mixed analysis of variance (ANOVA) with group JT09 and control as a between subjects variable and session/time as within subjects variables. Post hoc comparison to control for family wise error were conducted with Dunnet's or Sidak's when appropriate.

Results

Screening of Potential KOAs.

As a biological screen, the potential for oral availability of the eight peptides in our second generation library were evaluated in the acetic acid-induced writhing assay for peripheral pain. Rats were administered the various compounds by oral gavage at a screening dose of 20 mg/kg 20 min before i.p. injection with 2 ml/kg of 3% acetic acid. Ten minutes later, animals were assessed for writhing as described in the methods section. As shown in FIG. 8, several of the JT Pharma compounds (JT07, JT09, and JT22) were able to significantly block outward physical signs of peripheral pain in this model. Thus, several of the Arg modifications can impart CR665 with the ability to cross the gut barrier. In addition, JT07 and JT09 were statistically significantly different from saline when assessing the percentage of time writhing (Student's t-test, p<0.05), and were indistinguishable in potency when compared to morphine (Student's t-test, p<0.05). JT07 and JT09 were selected for further analysis.

Screening of CNS-Mediated Pain in KOAs.

As an initial screen for analgesic activity, JT07 and JT09 were evaluated in the hotplate analgesic model using a standard oral dose of 20 mg/kg. Percent maximum possible effect was analyzed for each compound and compared to morphine (10 mg/kg, i.p.). JT07 and JT09 did not show analgesic activity and were significantly different from morphine (Student's t-test, p<0.05).

Self-Administration.

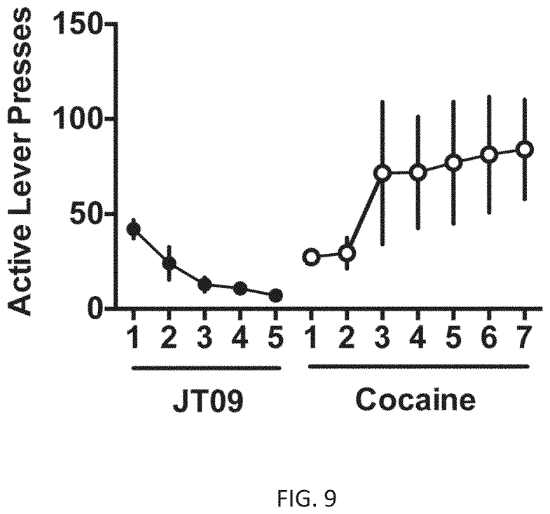

In an operant self-administration procedure in which rats are required to press a lever to receive an intravenous drug infusion, JT09 failed to maintain lever responding in rats over a five-day period (FIG. 9). The number of infusions decreased on all 4 days compared to day one of JT09 administration [F(4,28)=9.04, p<0.0001, and Dunnett post hoc, p<0.05]. Further, to ensure that these rats were not deficient in reward processing, we replaced JT09 with cocaine using a nose-poke operandi and the number of cocaine infusions increased over the 7 days [F(6,42)=4.6, p<0.0012] with significantly more infusion on days 6 and 7 (Dunnett post hoc, p<0.05).

Conditioned Place Preference.

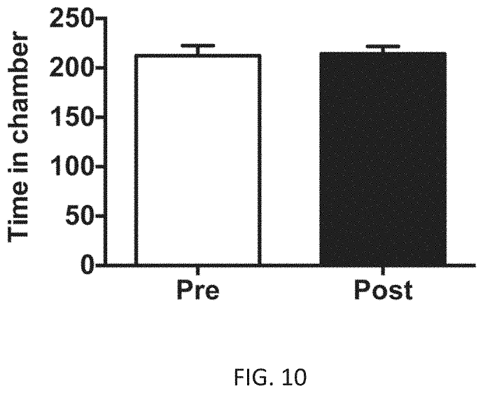

To further establish that JT09 does not have rewarding properties, rats were tested for the amount of time spent in an environment previously associated with JT09 compared to a saline paired compartment. After receiving alternate treatment of JT09 and saline over the course of eight days, rats did not develop a preference for JT09 over saline (FIG. 10).

Forced Swim.

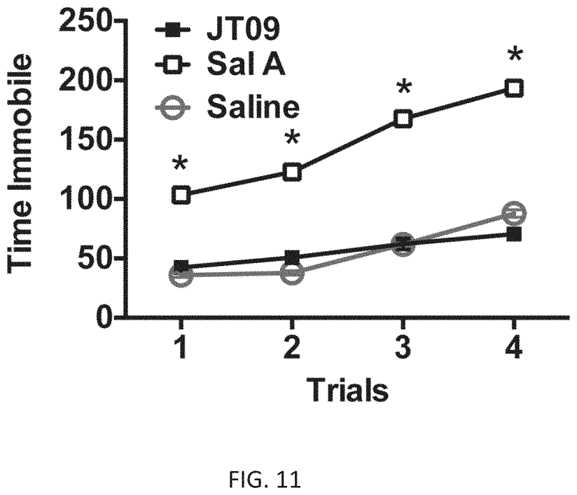

As an initial screening for dysphoria, rats were repeatedly tested in the forced swim assay. Saline and JT09 were statistically indistinguishable in all time bins (Student's t-test, p<0.05). There was a significant interaction between Sal A and JT09 [FIG. 11, F(3,30)=117, p<0.0001], specifically rats treated with JT09 had lower amounts of time spent immobile relative to Sal A during all trials (Sidak's multiple comparison, p<0.05). Further, the main effect of treatment [F(1,10)=947, p<0.0001] and time [F(3,30)=418, p<0.0001] were also significant.

Locomotor Activity.

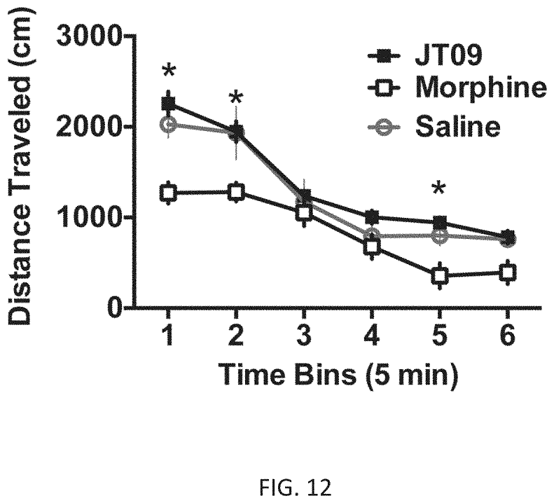

As a measure of sedation, rats were placed in activity chambers and distance traveled was evaluated. Saline and JT09 were statistically indistinguishable in all time bins (Student's t-test, p<0.05). There was a significant interaction between morphine and JT09 [FIG. 12, F(5,70)=7.0, p<0.0001], specifically JT09 had higher locomotor activity relative to morphine during time bins 1, 2, and 5 (Sidak's multiple comparison, p<0.05). Further, the main effect of treatment [F(1,14)=18.6, p<0.0007] and time [F(5,70)=84, p<0.0001] were also significant.

Maximum Tolerated Dose Determination.

The maximum tolerated dose (MTD) is used to determine the highest dose of JT09 that can be administered without promoting unacceptable side-effects. A maximum administered dose (MAD) of three times the EC90 of JT09 (90 mg/kg) did not produce mortality, a loss of more than 10% body weight, or overt signs of toxicity.

Multiple Dose Study.

Following the 14-day multiple dose study, six rats were submitted for necropsy. Upon necropsy, the lungs displayed a slight grey discoloration. The grey discoloration of the lungs may be due to the hemorrhage, which was seen histologically. Hemorrhage is a common change seen due to euthanasia with CO₂. The heart, stomach, intestines, liver, spleen, pancreas, kidneys, adrenal glands, skeletal muscle, bone, reproductive organs, and brain were all grossly normal.

### DISCUSSION

The lead compound, JT09, binds potently to activate the kappa-opioid receptor with an EC50 of 29.9 nM, while having an agonist selectivity for kappa—over both mu- and delta-opioid receptors of minimally >33,400 and likely much greater. Most importantly, JT09 exhibits oral activity at a sufficiently potent EC₅₀ of 4.7 mg/kg, while having a peripheral versus central selectivity of at least 900-fold. Both JT09 and morphine exhibit comparable analgesic activity in the acetic acid-induced writhing model for peripheral pain. However, in the hot plate model of centrally mediated pain, morphine was a potent analgesic, while JT09 exhibited no analgesic effects at the highest concentrations we were able to test. This indicates that administration of JT09 does not significantly attenuate centrally-mediated pain as it is unable to cross the blood brain barrier. Other potential centrally mediated side effects of morphine, which combine to make it a less than ideal drug, also were evaluated with JT09. Two different testing methods were utilized to examine the abuse liability of JT09. The most reliable test of abuse liability is a contingent drug self-administration model, in which rats are trained to press a lever for intravenous drug delivery. JT09 failed to maintain lever responding in rats over a five-day period, with the number of infusions decreasing over the last four days in comparison to day one. Positive controls demonstrated that the rats were not deficient in reward processing as cocaine administration resulted in the expected increase in the number of lever presses over the seven-day period of the procedure. In addition, we employed a conditioned place preference model to ensure that JT09 does not exhibit rewarding properties. As anticipated, rats did not develop a preference for JT09 over saline, thus further establishing that JT09 lacks rewarding properties.

Addiction is another major issue associated with centrally mediated analgesics such as morphine. Evaluation of early KOAs, including non-peptidic compounds U50,488, enadoline, ADL 10-0101, and ADL 10-0116, demonstrated a poor peripheral versus central distribution that resulted in centrally mediated sedation and dysphoria, which forced discontinuation of development. Thus, we employed two experiments to test for the induction of dysphoria and sedation. To test for the promotion of dysphoria, we examined rats in the forced swim assay, comparing JT09 to salvinorin A (a centrally-active KOA). Dysphoria is measured in the amount of time spent immobile during the last four minutes of each test trial. Administration of JT09 did not induce more than a baseline time of immobility, in contrast to rats administered salvinornin A. As a measure of sedation, we used locomotor boxes to determine the activity levels of rats after receiving JT09 and morphine, which is highly sedative as a result of its ability to act centrally. There was a significant difference between morphine- and JT09-treated rats, specifically JT09 showed no sedation, whereas morphine was highly sedative. Thus, JT09 does not appear to elicit sedation due to its high peripheral selectivity, making the CR665 derivative JT09 successfully improved over early KOAs and morphine by elimination of its ability to induce centrally mediated effects.

In conclusion, this study provides data to demonstrate that JT09 is orally active and peripherally restricted, with the potential for clinical and out-patient use as an analgesic. The EC50 of JT09 is at a druggable level for an oral analgesic, and appears as efficacious as morphine in alleviating peripheral pain. In addition, JT09 does not promote the negative CNS-mediated effects associated with morphine, including sedation, dysphoria, tolerance, and addiction.

### Example 3: Preparation of Implantable Devices

Implantable devices will be prepared using an extrusion process in a Microtruder device (Rancastle, RC-025-CF-RF). In order to facilitate feeding into the extruder and to enable mixing of kappa-opioid agonists and other substances to be incorporated into the implants, EVA is ground into smaller particle sizes prior to extrusion. The extrusion process is performed under argon gas to prevent oxidation of kappa-opioid agonists, if needed. All blends of copolymer and drug(s) are prepared by rolling in a 120 ml amber bottle for approximately 10 minutes. The blend is then fed through the Microtruder. Parameters used for extrusion are known in the art.

All of the materials used during the extrusion process are protected from light to prevent light-catalyzed oxidation. The extruder is set to the required temperatures and allowed to reach equilibrium. After the extruder has reached equilibrium, approximately 15 grams of blend are extruded and cut into 18-inch rods. The diameter is measured at 2.4 mm. The rods are then cut to the desired implant length of 26 mm. The implants are then washed by placing them on an aluminum screen and immersing them in ethanol (approximately 50 ml per implant). The implants are washed for approximately 30, 60, or 120 minutes in the ethanol bath. The washed implants are air dried for 10 minutes and oven dried at 40° C. for 1 hour before drying in a vacuum over for 24 hours at 30° C. The implants are packaged into 20 ml glass vials in the presence of argon gas, sealed, and then sterilized by gamma irradiation.

What is claimed is:

1. A sustained release composition comprising a biocompatible polymeric matrix and a kappa-opioid receptor agonist of formula I: 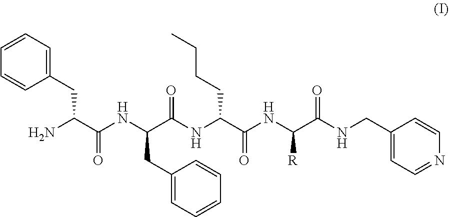
or pharmaceutically acceptable salts, solvates, and stereoisomers thereof
wherein R is: 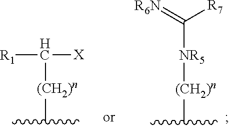
wherein n is an integer from 1 to 4;
X is —NR₂R₃ or —N^{⊕}R₂R₃R₄;
each of R₁, R₂, R₃, R₄, is independently, hydrogen, C₁-C₅ alkyl, C₁-C₅ substituted alkyl, C₁-C₅ alkenyl, C₁-C₅ substituted alkenyl, C₁-C₅ alkynyl, C₁-C₅ substituted alkynyl, cycloalkyl, aryl, substituted aryl, or arylalkyl;
R₇ is hydrogen, C₁-C₅ alkyl, C₁-C₅ substituted alkyl, C₁-C₅ alkenyl, C₁-C₅ substituted alkenyl, C₁-C₅ alkynyl, C₁-C₅ substituted alkynyl, cycloalkyl, aryl, substituted aryl, arylalkyl, or and —NR₈R₉;
each of R₅, R₆, R₈, R₉, is independently, hydrogen, C₁-C₅ alkyl, C₁-C₅ substituted alkyl, C₁-C₅ alkenyl, C₁-C₅ substituted alkenyl, C₁-C₅ alkynyl, C₁-C₅ substituted alkynyl, cycloalkyl, aryl, substituted aryl, or arylalkyl; or
alternatively, R₅ and R₉ taken together with the nitrogen atom to which they are attached form a heterocyclic ring; or
alternatively, R₆ and R₉ taken together with the nitrogen atom to which they are attached form a heterocyclic ring.

2. The sustained release composition of claim 1, wherein R is:

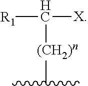

3. The sustained release composition of claim 2, wherein R is:

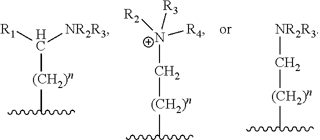

4. The sustained release composition of claim 1, wherein R is:

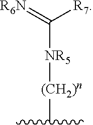

5. The sustained release composition of claim 4, wherein R is:

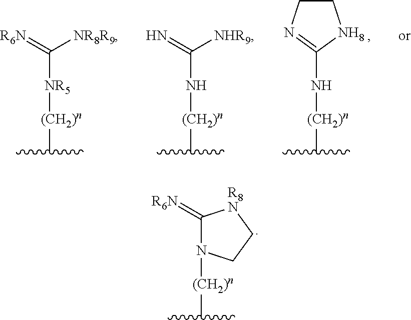

6. The sustained release composition of claim 4, wherein R is:

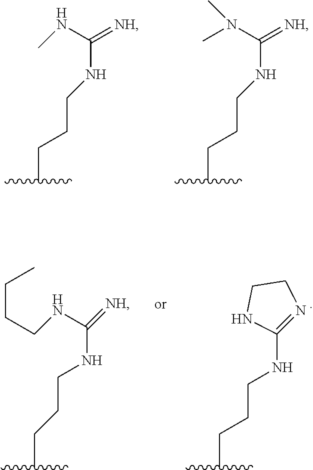

7. The sustained release composition of claim 2, wherein R is:

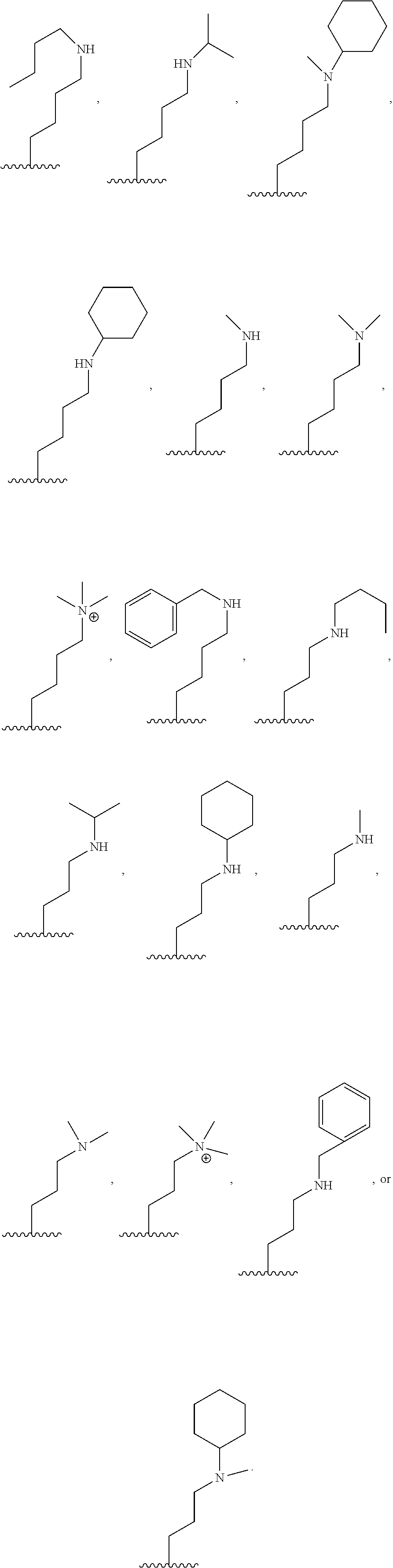

8. The sustained release composition of claim 1, wherein the biocompatible polymeric matrix is ethylene vinyl acetate (EVA) copolymer, crosslinked poly(vinyl alcohol), poly(hydroxy ethylmethacrylate), acyl substituted cellulose acetates, hydrolyzed alkylene-vinyl acetate copolymers, polyvinyl chloride, polyvinyl acetate, polyvinyl alkyl ethers, polyvinyl fluoride, polycarbonate, polyurethane, polyamide, polysulphones, styrene acrylonitrile copolymers, crosslinked poly(ethylene oxide), poly(alkylenes), poly(vinyl imidazole), poly(esters), poly(ethylene terephthalate), polyphosphazenes, chlorosulphonated polyolefines, poly-lactides (PLA), poly-glycolides (PGA), or combinations thereof.

9. The sustained release composition of claim 8, wherein the EVA copolymer comprises about 33% vinyl acetate of the total weight of the copolymer.

10. The sustained release composition of claim 1, wherein the kappa-opioid agonist comprises about 10 to about 85% of the total weight of the composition.

11. The sustained release composition of claim 1, wherein the biocompatible polymer matrix is a rod shaped implantable device having a diameter of about 0.5 mm to about 10 mm, and a length of about 0.5 cm to about 10 cm.

12. The sustained release composition of claim 1, wherein the composition releases about 0.1 mg to about 10 mg of the kappa-opioid agonist per day.

13. The sustained release composition of claim 1, wherein the composition releases the kappa-opioid agonist for about 1 week to about 24 months.

14. A method of treating chronic pain in a subject, comprising administering to the subject a sustained release composition comprising a biocompatible polymeric matrix and a kappa-opioid agonist of formula I: 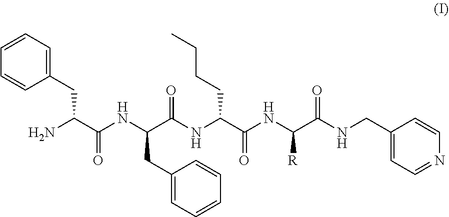
or pharmaceutically acceptable salts, solvates, and stereoisomers thereof
wherein R is: 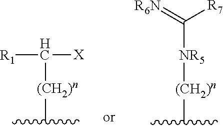
wherein n is an integer from 1 to 4;
X is —NR₂R₃ or —N^{⊕}R₂R₃R₄;
each of R₁, R₂, R₃, R₄, is independently, hydrogen, C₁-C₅ alkyl, C₁-C₅ substituted alkyl, C₁-C₅ alkenyl, C₁-C₅ substituted alkenyl, C₁-C₅ alkynyl, C₁-C₅ substituted alkynyl, cycloalkyl, aryl, substituted aryl, or arylalkyl;
R₇ is hydrogen, C₁-C₅ alkyl, C₁-C₅ substituted alkyl, C₁-C₅ alkenyl, C₁-C₅ substituted alkenyl, C₁-C₅ alkynyl, C₁-C₅ substituted alkynyl, cycloalkyl, aryl, substituted aryl, arylalkyl, or —NR₈R₉;
each of R₅, R₆, R₈, R₉, is independently, hydrogen, C₁-C₅ alkyl, C₁-C₅ substituted alkyl, C₁-C₅ alkenyl, C₁-C₅ substituted alkenyl, C₁-C₅ alkynyl, C₁-C₅ substituted alkynyl, cycloalkyl, aryl, substituted aryl, or arylalkyl; or
alternatively, R₅ and R₉ taken together with the nitrogen atom to which they are attached form a heterocyclic ring; or
alternatively, R₆ and R₉ taken together with the nitrogen atom to which they are attached form a heterocyclic ring; and
wherein the sustained release composition releases a therapeutically effective amount of the kappa opioid agonist over a sustained period of time.

15. The method of claim 14, wherein the chronic pain is peripheral pain, visceral pain, thermal pain, bone pain, neuropathic pain, chronic low back pain, inflammatory pain, and pain associated with cancer, fibromyalgia, irritable bowel syndrome, chronic arthropathy, post herpetic neuralgia, trigeminal neuralgia, migraine, refractory angina pectoris (chest pains), interstitial cystitis (inflammation around bladder) or combinations thereof.

16. The method of claim 14, wherein the sustained release composition is administered by a depot injection or by implant.

17. The method of claim 14, wherein the sustained release composition is a rod shaped implantable device having a diameter of about 0.5 mm to about 10 mm, and a length of about 0.5 cm to about 10 cm.

18. The method of claim 14, wherein the biocompatible polymeric matrix is ethylene vinyl acetate (EVA) copolymer, crosslinked poly(vinyl alcohol), poly(hydroxy ethylmethacrylate), acyl substituted cellulose acetates, hydrolyzed alkylene-vinyl acetate copolymers, polyvinyl chloride, polyvinyl acetate, polyvinyl alkyl ethers, polyvinyl fluoride, polycarbonate, polyurethane, polyamide, polysulphones, styrene acrylonitrile copolymers, crosslinked poly(ethylene oxide), poly(alkylenes), poly(vinyl imidazole), poly(esters), poly(ethylene terephthalate), polyphosphazenes, chlorosulphonated polyolefines, poly-lactides (PLA), poly-glycolides (PGA), or combinations thereof.

19. The method of claim 14, wherein the composition releases the kappa-opioid agonist for about 1 week to about 24 months.

20. The method of claim 14, wherein the composition releases about 0.1 mg to about 10 mg of kappa-opioid agonist per day.

21. The method of claim 14, wherein the kappa-opioid agonist of formula I is highly specific for kappa-opioid receptors with little or no agonist or antagonist activity to mu or delta opioid receptors.
